(19) United States
(12) Patent Application Publication
(10) Pub. No.: US 2022/0267780 A1
(43) Pub. Date: Aug. 25, 2022

(54) CONJUGATES OF BILE ACIDS AND THEIR DERIVATIVES FOR ACTIVE MOLECULES DELIVERY

(71) Applicant: FINICE - S.R.L., Reggio Emilia (IT)

(72) Inventors: Beniamino Palmieri, Modena (IT); Matteo Bovolenta, Vigarano Pieve (IT); Paola Braghetta, Padova (IT); Massimo Luigi Capobianco, Bologna (IT); Elena Marchesi, Ferrara (IT); Alessandro Medici, Bologna (IT); Sibilla Molon, Lonigo (IT); Daniela Perrone, Ferrara (IT); Paola Rimessi, Ferrara (IT)

(21) Appl. No.: 17/287,416

(22) PCT Filed: Oct. 22, 2019
(86) PCT No.: PCT/IB2019/059014

§ 371 Date: Oct. 28, 2021

(30) Foreign Application Priority Data
Oct. 22, 2018 (IT) 102018000009682

(51) Int. Cl. C12N 15/113; A61K 47/54; A61P 21/00

(52) U.S. Cl. CPC C12N 15/1138; A61K 47/554; A61P 21/00; C12N 2310/14

(57) ABSTRACT

A conjugate of oligonucleotides and bile acid derivatives having the structure (I), (II) or (III), pharmaceutical compositions thereof, and uses thereof are described.

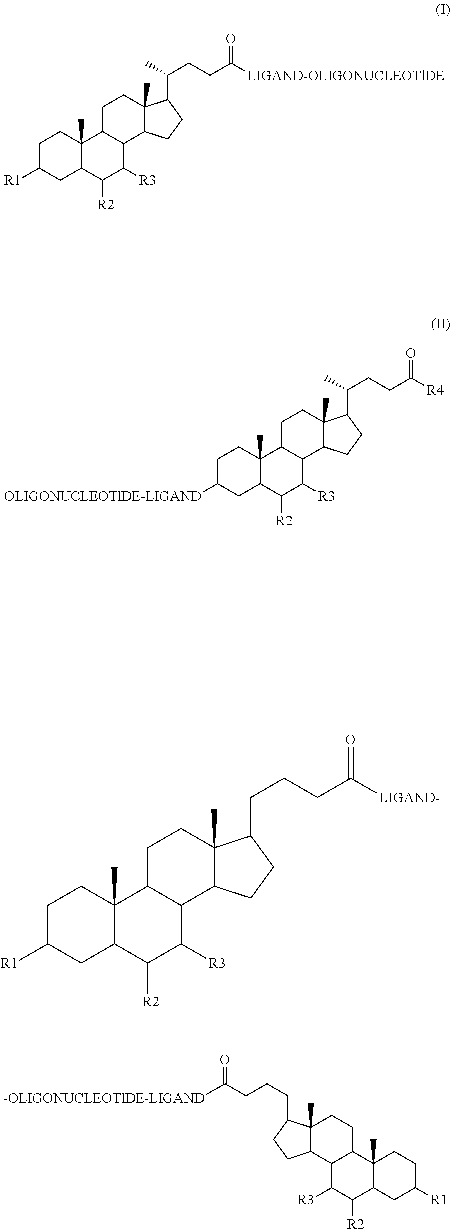

### CROSS-REFERENCE TO RELATED APPLICATIONS

This patent application is a U.S. national phase of International Application No. PCT/IB2019/059014, filed Oct. 22, 2019, which claims priority from Italian patent application no. 102018000009682 filed on Oct. 22, 2018, the entire disclosure of which is incorporated herein by reference.

### INCORPORATION BY REFERENCE OF SEQUENCE LISTING

This application contains, as a separate part of the disclosure, a Sequence Listing in computer-readable form (filename: 56610_Seqlisting.txt; Size 2,136 bytes; created Apr. 21, 2021), which is incorporated by reference in its entirety.

### FIELD OF THE INVENTION

The present invention refers to conjugates of bile acids or their derivatives with oligonucleotides, in particular for the treatment of Duchenne muscular dystrophy.

### STATE OF THE PRIOR ART

Duchenne muscular dystrophy (DMD) is the most widespread fatal genetic disorder, occurring in one case every 5000 males. It entails muscular degeneration with loss of mobility and premature death. This disease is caused by the deletion of one or more exons of the dystrophin gene with interruption of the gene reading frame and consequent total loss of the functional protein expression. The dystrophin gene is located on the X chromosome and is recessive. Therefore, only males are affected by this disease while females can be healthy carriers without symptoms. The dystrophin is located in the muscle on the cytoplasmic face of the sarcolemma where it interacts with the F-actin of the cytoskeleton. This protein is furthermore associated with a sarcolemmal protein complex known as dystrophin associated proteins (DAPs) and dystrophin associated glycoproteins (DAGs). The lack of the dystrophin leads to a loss of the DAPs and to rupture of the dystroglycan-protein complex; this rupture makes the sarcolemma susceptible to lacerations during muscle contraction.

Duchenne muscular dystrophy is usually recognized at the age of three although approximately half of the patients shows signs of the disease before they begin to walk. The first symptoms include the inability to walk or run when these functions should already have been acquired; or even when these abilities have been acquired, children appear less reactive and tend to fall easily. Over time, the difficulties increase, for example in walking, running and climbing stairs. The tendon reflexes are first reduced and then disappear parallel to loss of the muscle fibres; the last to disappear are the Achilles reflexes. The bones become thin and demineralized. The smooth muscles are spared, while the heart is affected and various types of arrhythmia can appear. Death is usually due to respiratory failure, lung infection or heart failure. Life expectancy always depends on the individual patient; in the last ten years life expectancy has significantly increased due to overnight ventilation.

Due to the genetic nature of the disease, the gene therapy is a promising option for the treatment of DMD.

There are many therapeutic approaches in DMD aimed at limiting the dystrophic process and at increasing the muscular regeneration processes. To date, one of the therapies being studied is exon skipping, a technique that acts directly at the level of the messenger RNA, with the use of antisense oligonucleotides (AON). The antisense oligonucleotides (AONs) are small chemically modified molecules of RNA, which can be used to modulate the splicing and re-establish the gene reading frame that encodes for dystrophin. In fact DMD is caused, as we have said, by the deletion of one or more exons of the dystrophin gene, with interruption of the gene reading frame and consequent loss of the functional protein expression. The mutations that maintain the gene reading frame lead to the formation of a protein with internal deletions but partially functional, and are associated with a less serious dystrophy phenotype called Becker Muscular Dystrophy (BMD). The use of these oligonucleotides interferes with the splicing signals and induces the skipping of specific exons in the pre-mRNA of the DMD gene, thus restoring the gene reading frame. This allows the production of a partially functioning dystrophin and the conversion of a severe Duchenne dystrophy into the phenotype attributable to Becker muscular dystrophy. Many in vivo studies are reported in literature, which have confirmed the broad therapeutic applicability of exon skipping. The main studies have been conducted using cell cultures derived from patients with different mutations, but above all by exploiting the availability of murine models of Duchenne, in particular mdx mice, without dystrophin due to a nonsense mutation of exon 23. In particular, the intramuscular administration of antisense oligonucleotides directed towards the mutated exon 23 restores the expression of the dystrophin for at least 3 months. In recent years, various in vivo studies have been reported, in which the AONs were administered intramuscularly, intraperitoneally, intravenously and orally.

Nevertheless, the search for systems that can improve the effectiveness of oligonucleotide administration is ongoing.

The object of the present invention is to solve the technical problems previously mentioned.

In particular, an object of the present invention is to provide new conjugates of oligonucleotides that allow an improvement in administration effectiveness.

### SUBJECT OF THE INVENTION

The object of the present invention is achieved by a conjugate according to claim 1, by a pharmaceutical composition thereof according to claim 11 and by the uses thereof according to claims 12 and 13.

The following paragraphs provide the definitions of the various chemical fractions of the compounds according to the invention and are intended to be applied uniformly in the entire specification and in the claims unless an expressly illustrated definition otherwise provides a broader definition.

The term “alkyl”, as used herein, refers to saturated aliphatic hydrocarbon groups. Said term includes linear (non-branched) chains or branched chains.

Non-limiting examples of alkyl groups according to the invention are, for example, methyl, ethyl, propyl, isopropyl, n-butyl, iso-butyl, tert-butyl, n-pentyl, iso-pentyl, n-hexyl and similar.

The term “pharmaceutically acceptable salts” refers to salts of the compounds of Formula (I), (II) or (III) identified below which maintain the desired biological activity and have been accepted by the regulatory authorities.

As used herein, the term “salt” refers to any salt of a compound according to the present invention prepared from an inorganic or organic acid or base and salts formed internally. Typically, said salts have a physiologically acceptable anion or cation.

Moreover, the compounds of Formula (I), (II) or (III) can form an acid addition salt or a salt with a base, according to the type of substituent, and said salts are included in the present invention on the condition that they are pharmaceutically acceptable salts.

Examples of said salts include, but are not limited to, acid addition salts formed with inorganic acids, salts formed with organic acids.

The compounds of Formula (I), (II) or (III) containing acid protons can be converted into their therapeutically active non-toxic base addition salt forms, for example metal or amine salts, by means of treatment with appropriate organic and inorganic bases.

Physiologically or pharmaceutically acceptable salts are particularly suitable for medical applications due to their greater solubility in water than the original compound.

Pharmaceutically acceptable salts can also be prepared from other salts including other pharmaceutically acceptable salts of the compounds of Formula (I), (II) or (III) using conventional methods.

Experts in organic chemistry techniques will appreciate that many organic compounds can form complexes with solvents in which they are made to react or from which they are precipitated or crystallized. These complexes are known as “solvates”. For example, a complex with water is known as a “hydrate”. The solvates of the compounds of the invention fall within the scope of the invention. The compounds of Formula (I) can be easily isolated in association with the molecules of solvent by means of crystallization or evaporation of an appropriate solvent to give the corresponding solvates.

The compounds of Formula (I), (II) or (III) can be in crystalline form. In some embodiments, the crystalline forms of the compounds of Formula (I), (II) or (III) are polymorphic.

The present invention also includes isotopically labelled compounds, which are identical to those described in Formula (I), (II) or (III) and following, but differ due to the fact that one or more atoms are substituted by an atom having an atomic mass or mass number different from the atomic mass or mass number usually found in nature. Examples of isotopes that can be incorporated in the compounds of the invention and the relative pharmaceutically acceptable salts include isotopes of hydrogen, carbon, nitrogen and oxygen, such as ²H, ³H, ¹¹C, ¹³C, ¹⁴C, ¹⁵N, ¹⁷O, ¹⁸O.

The compounds of the present invention and the pharmaceutically acceptable salts of said compounds containing the above-mentioned isotopes and/or other isotopes of other atoms fall within the scope of the present invention. Isotopically labelled compounds of the present invention, for example those in which the radioactive isotopes are incorporated such as ³H, ¹⁴C, are useful in tissue distribution assays of drugs and/or substrates.

Tritium isotopes, i.e. ³H and carbon-14, namely ¹⁴C, are particularly preferred due to their ease of preparation and detectability. The isotope ¹¹C is particularly useful in Positron Emission Tomography (PET). Moreover, substitution with heavier isotopes such as deuterium, namely ²H, can produce certain therapeutic advantages resulting from greater metabolic stability, for example increased half-life in vivo or reduced dosing requirements and therefore may be preferred in some circumstances. Isotopically labelled compounds of Formula (I) of this invention can generally be prepared by carrying out the procedures illustrated in the diagrams and/or in the examples below, by substituting a non-isotopically labelled reagent with a readily available isotopically labelled reagent.

Certain groups/substituents included in the present invention can be present as isomers. Consequently, in some embodiments, the compounds of Formula (I), (II) or (III) can have asymmetric carbon atoms or axial asymmetries in some cases and, correspondingly, they can exist in the form of optical isomers such as a form (R), a form (S), and similar. The present invention includes within its scope all said isomers, including racemates, enantiomers and relative mixtures.

In particular, the scope of the present invention includes all the stereoisomeric forms, including enantiomers, diastereoisomers and relative mixtures, including racemates and the general reference for the compounds of Formula (I) includes all the stereoisomeric forms, unless indicated otherwise.

In general, the compounds or salts of the invention must be interpreted in such a way as to exclude those compounds (if present) which are chemically unstable, both per sé or in water, which are not clearly suitable for pharmaceutical use through all the oral, parenteral or other administration methods. Said compounds are known to chemists skilled in the art.

### BRIEF DESCRIPTION OF THE DRAWINGS

The invention will now be described with reference to the attached figures, provided purely by way of non-limiting example, in which:

FIG. 1 illustrates the concentrations of RNA extracted from myotubes obtained by differentiation of a cell line of human myoblasts immortalized with deletion of the exon 52 of the dystrophin gene (DMD).

FIG. 2 illustrates the results of the in vitro study of the skipping efficiency of the conjugates of the invention.

FIG. 3 illustrates A) RT-PCR with primer able to amplify the dystrophin transcript between the exons 50 and 54 in untreated (UT) myotubes and treated with oligonucleotide PRO051 or with the conjugated oligonucleotides 9 and 17; B) percentage and normalized quantification of the skipping induced by the antisense oligonucleotides directed against the exon 51 of the DMD gene; C) immunofluorescence analysis of the dystrophin (arrows) in immortalized myogenic cells derived from a patient having deleted the exon 52 of the dystrophin gene.

FIG. 4 illustrates the exon skipping of the exon 2 of the dystrophin gene. The graph at the top shows the position and sequence of the antisense oligonucleotides used. The histogram shows the skipping percentages.

FIG. 5 illustrates the skipping values obtained with the compound 22 and the oligonucleotide with SEQ ID No. 2 in the diaphragm, in the gastrocnemius muscle and in the heart.

FIG. 6 illustrates the quantification of the dystrophin protein in the muscles of mdx mice treated for 12 weeks.

FIG. 7 illustrates the body weight trend of mdx mice treated intraperitoneally with the oligonucleotides 22 and SEQ ID No.2 for 12 weeks, with respect to the mdx control mice (mdx PBS).

FIG. 8 illustrates the results of the test performed to verify motor coordination and neuromuscular strength of mdx mice treated intraperitoneally with the oligonucleotides 22 and SEQ ID No.2 for 12 weeks, with respect to the mdx control mice (mdx PBS).

FIG. 9 illustrates A) the values of the anterior tibial median area in cross-section (CSA) of the fibres in the mdx mice treated with the compound 22 and with SEQ ID No.2 with respect to the mdx control mice (mdx PBS); B) images showing hematoxylin-eosin in mdx mice treated for 12 weeks with PBS, compound 22 and with SEQ ID No.2 intraperitoneally; C) analysis of the percentage of necrotic fibres out of the total number of fibres. *, p<0.05; **, p<0.01; ns, non-significant.

FIG. 10 illustrates A) the values of the median area of the gastrocnemius muscle in cross-section (CSA) of the fibres in mdx mice treated with the compound 22 and with SEQ ID No.2 with respect to the mdx control mice (PBS); B) images showing hematoxylin-eosin in mdx mice treated for 12 weeks with PBS, compound 22 and with SEQ ID No.2 intraperitoneally; C) analysis of the percentage of necrotic fibres out of the total number of fibres. *, p<0.05; **, p<0.01.

FIG. 11 illustrates A) the values of the median area of the diaphragm in cross-section (CSA) of the fibres in mdx mice treated with the compound 22 and with SEQ ID No.2 with respect to the mdx control mice (PBS); B) images showing hematoxylin-eosin in mdx mice treated for 12 weeks with PBS, compound 22 and with SEQ ID No.2 intraperitoneally; C) analysis of the percentage of necrotic fibres out of the total number of fibres. *, p<0.05; p<0.01.

FIG. 12 illustrates the results of the immunofluorescence analysis of the dystrophin (in red) expressed in the myofibers of A) anterior tibial muscle, B) gastrocnemius muscle, and C) diaphragm of mdx mice treated for 12 weeks with PBS, compound 22 and with SEQ ID No.2 intraperitoneally.

FIG. 13 illustrates chromatograms of the HPLC-MS/MS analysis of an equimolar mixture of the oligonucleotides 22 and relative Internal Standard 23. Various channels.

FIG. 14 illustrates chromatograms of the HPLC-MS/MS analysis of an equimolar mixture of the oligonucleotides SEQ ID 2 and relative Internal Standard SEQ ID 1. Various channels.

FIG. 15 illustrates the quantities of oligonucleotides 22 and SEQ ID 2 found intact in the various tissues examined by HPLC-MS/MS method, in different experiments. Ordered on a logarithmic scale.

FIG. 16 illustrates the quantities of oligonucleotides 22 and SEQ ID 2 found intact in the various tissues examined by HPLC-MS/MS method, in different experiments. Ordered on a linear scale.

### PREFERRED EMBODIMENT OF THE INVENTION

According to a first aspect of the invention, an oligonucleotide conjugate is provided with bile acid derivatives having structure (I) or (II) or (III)

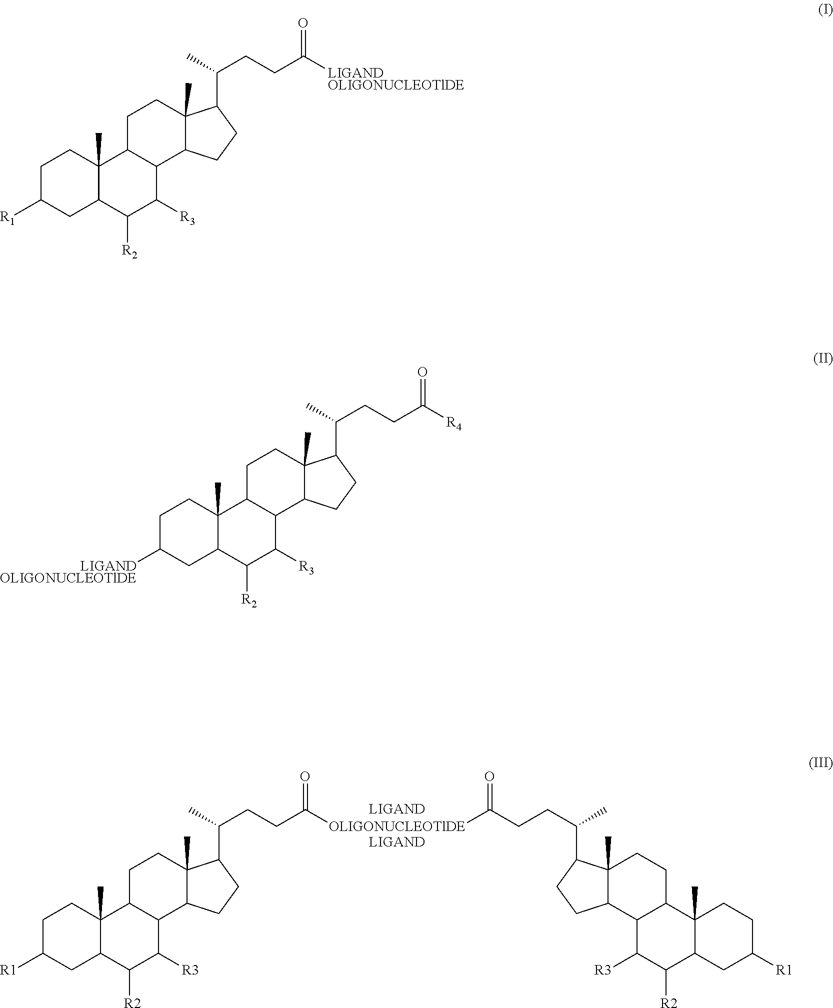

in which
R₁, R₂ and R₃ are independently selected from the group consisting of H, OH, NH₂, —NHC(O)R₅ and C(O)R₅;
R₄ is selected from the group consisting of OH, NH₂, —NH(C₁₋₆alkyl)SO₃H;
R₅ is selected from the group consisting of a linear or branched saturated or partially unsaturated C₃-C₃₁ aliphatic hydrocarbon;
the ligand has the formula (IV) or (V)

a) —X—Y—NH(C₂₋₁₀alkyl)OP(═O)(Z)O— (IV)

in which
X binds the bile acid residue and is selected from the group consisting of bond, —NHC(O)(C₂₋₁₀alkyl)C(O) and —NH(C₂₋₁₀alkyl(NHR₆))C(O)— where R₆ is selected from the group consisting of —H and

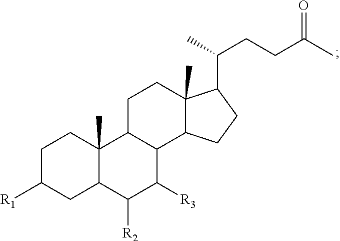

Y is selected from the group consisting of bond and NH(C₂₋₁₀alkyl)OC(O);
Z is selected from the group consisting of S⁻ and O⁻ and the group OP(═O)(Z)O— binds the oligonucleotide or
b)

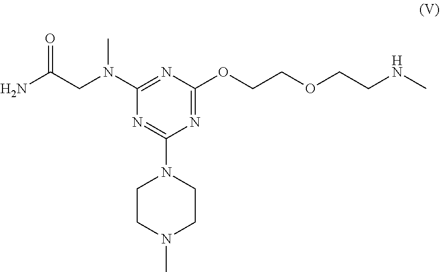 [SMILES: CNCCOCCOc1nc(N(C)CC(N)=O)nc(N2CCN(C)CC2)n1]

where the piperazine residue binds the oligonucleotide and the amine residue binds the bile acid residue.

In one embodiment, R₁ is selected from the group consisting of OH, NH₂, —NHC(O)R₅, preferably R₁ is selected from the group consisting of OH, NH₂, —NHC(O)(CH₂)₃(CH═CH—CH₂)₅CH₃ and —NHC(O)(CH₂)₂(CH═CH—CH₂)₆CH₃.

In a further embodiment, R₃ is selected from the group consisting of —H and —OH, in particular βOH.

In a further embodiment, R₄ is selected from the group consisting of OH and —NH(C₂H₄)SO₃H.

The ligand can be selected from the group consisting of

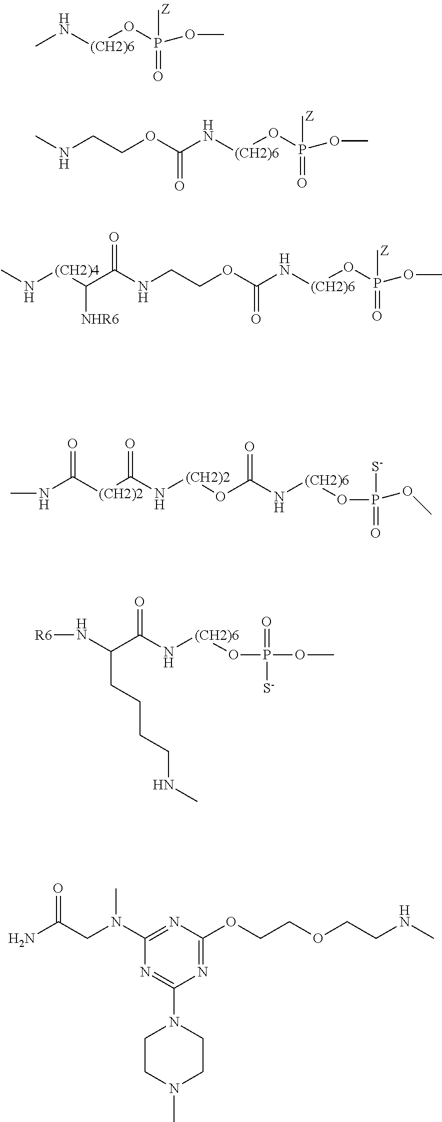

The oligonucleotide can be an antisense oligonucleotide specific for a splicing sequence in an mRNA of interest, in particular selected from the group consisting of SEQID No.1, SEQID No.2, SEQID No.3, SEQID No.4, SEQID No.5 and SEQID No.6 (Table 1).

TABLE 1

| SEQ | | | |
|---|---|---|---|
| ID | | | |
| No. | SEQUENCE 5′→3′ | | Common |
| 1 | ucaaggaagauggcauuucu | 2′OMe- | PRO051 |
| | | ribose | |
| 2 | ggccaaaccucggcuuaccu | 2′OMe- | M23D |
| | | ribose | Short |
| 3 | ggccaaaccucggcuuaccugaaau | 2′OMe- | M23D |
| | | ribose | |
| 4 | guuuucuuuugaacaucuucucuuuc | 2′OMe- | Long |
| | | ribose | |
| 5 | ccauuuugugaauguuuucuuuugaac | 2′OMe- | H2A |
| | auc | ribose | |
| 6 | ucaaggaagauggcauuucu | morph- | PMO |
| | | oline | |

The oligonucleotides can be bound to the ligand through their terminal 3′ or through the terminal 5′. Below, the wording

[symbol]

5′-SEQ ID No. n-3′

indicates an oligonucleotide having one of the sequences indicated in table 1 (namely in which n is 1-6) bound to the ligand through its own end 5′.
Likewise, the wording

[symbol]

3′-SEQ ID No. n-5′

indicates an oligonucleotide having one of the sequences indicated in table 1 (namely in which n is 1-6) bound to the ligand through its own end 3′.

Preferably, the conjugates according to the invention are selected from the group consisting of:

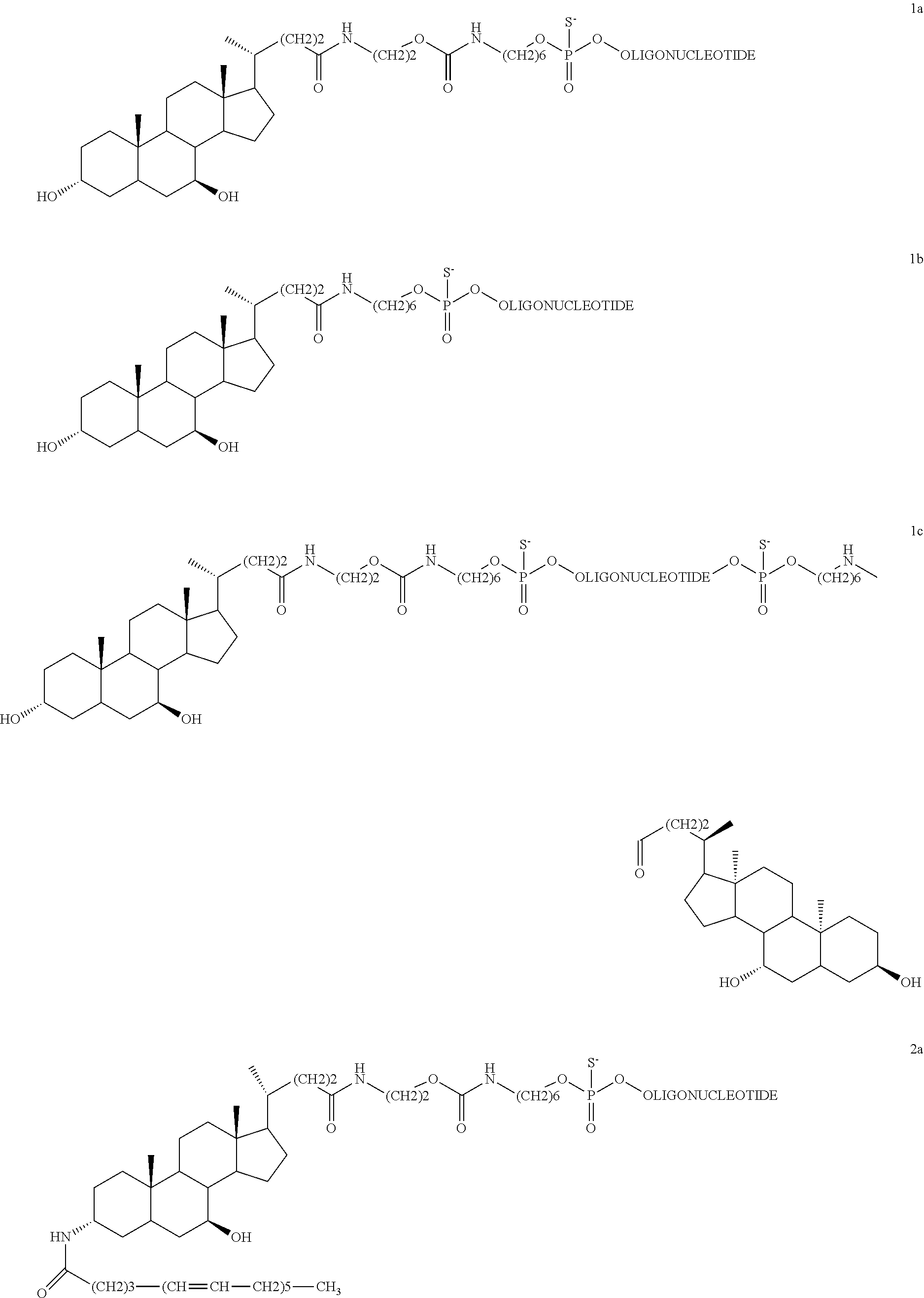

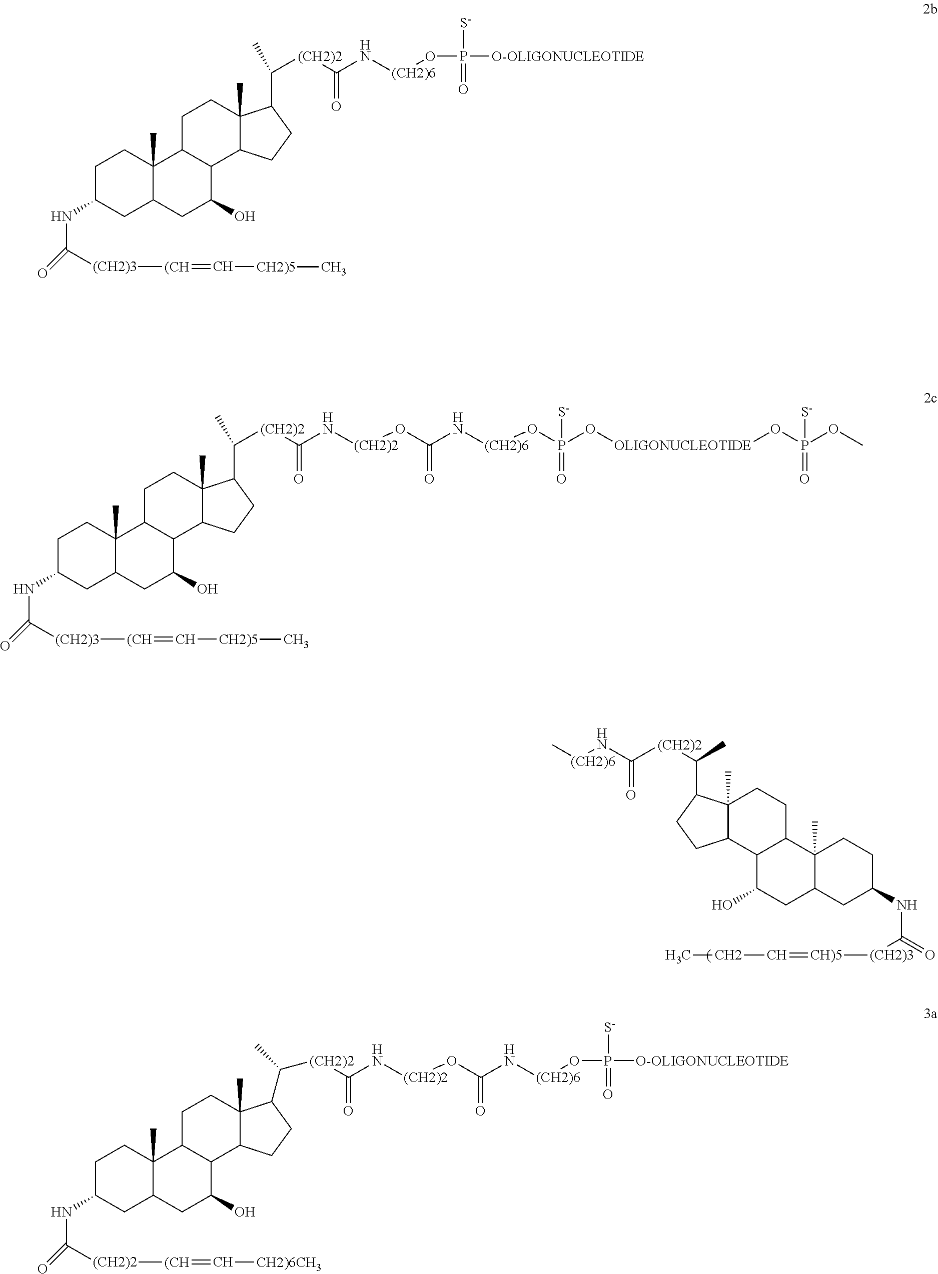

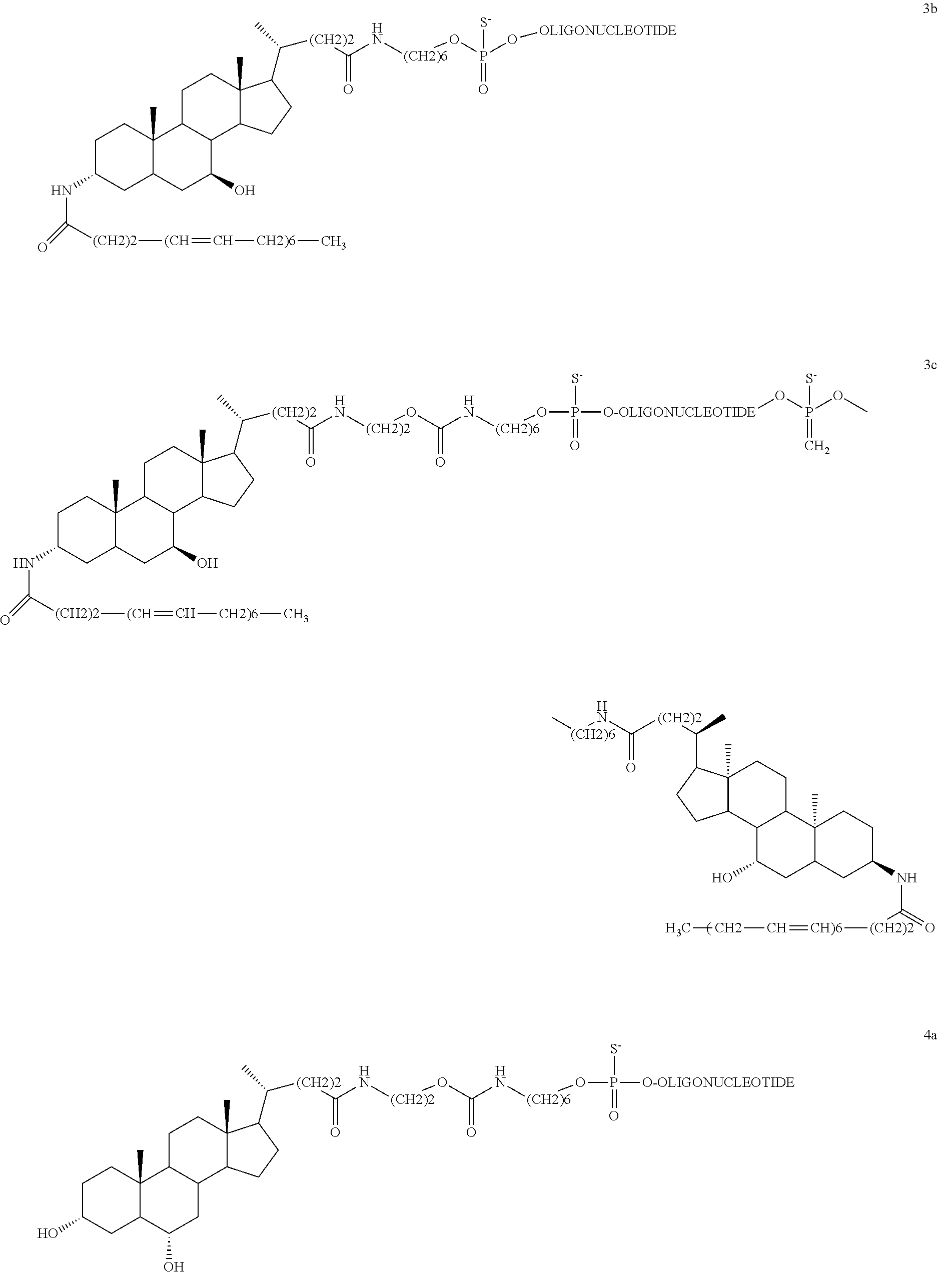

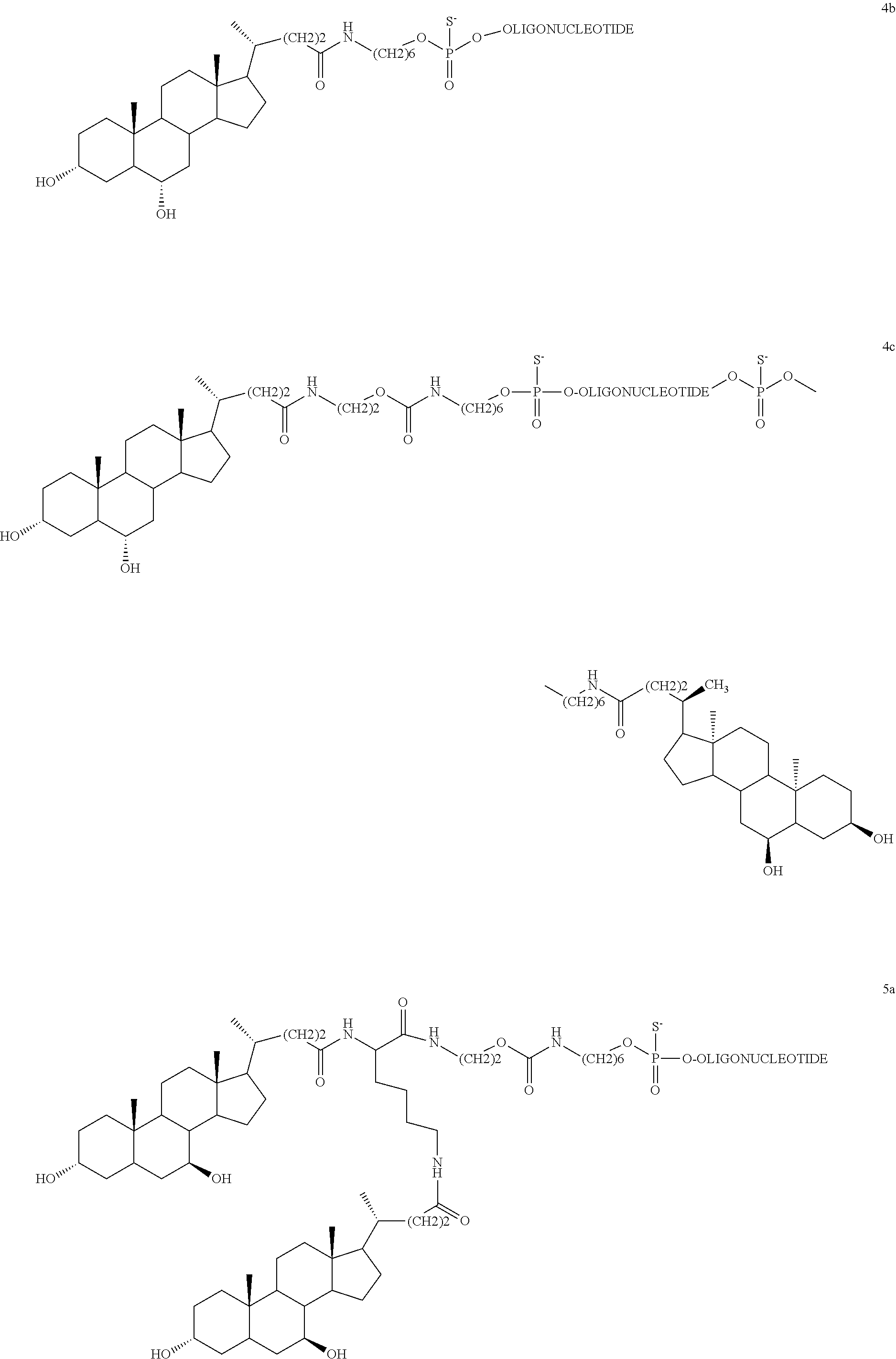

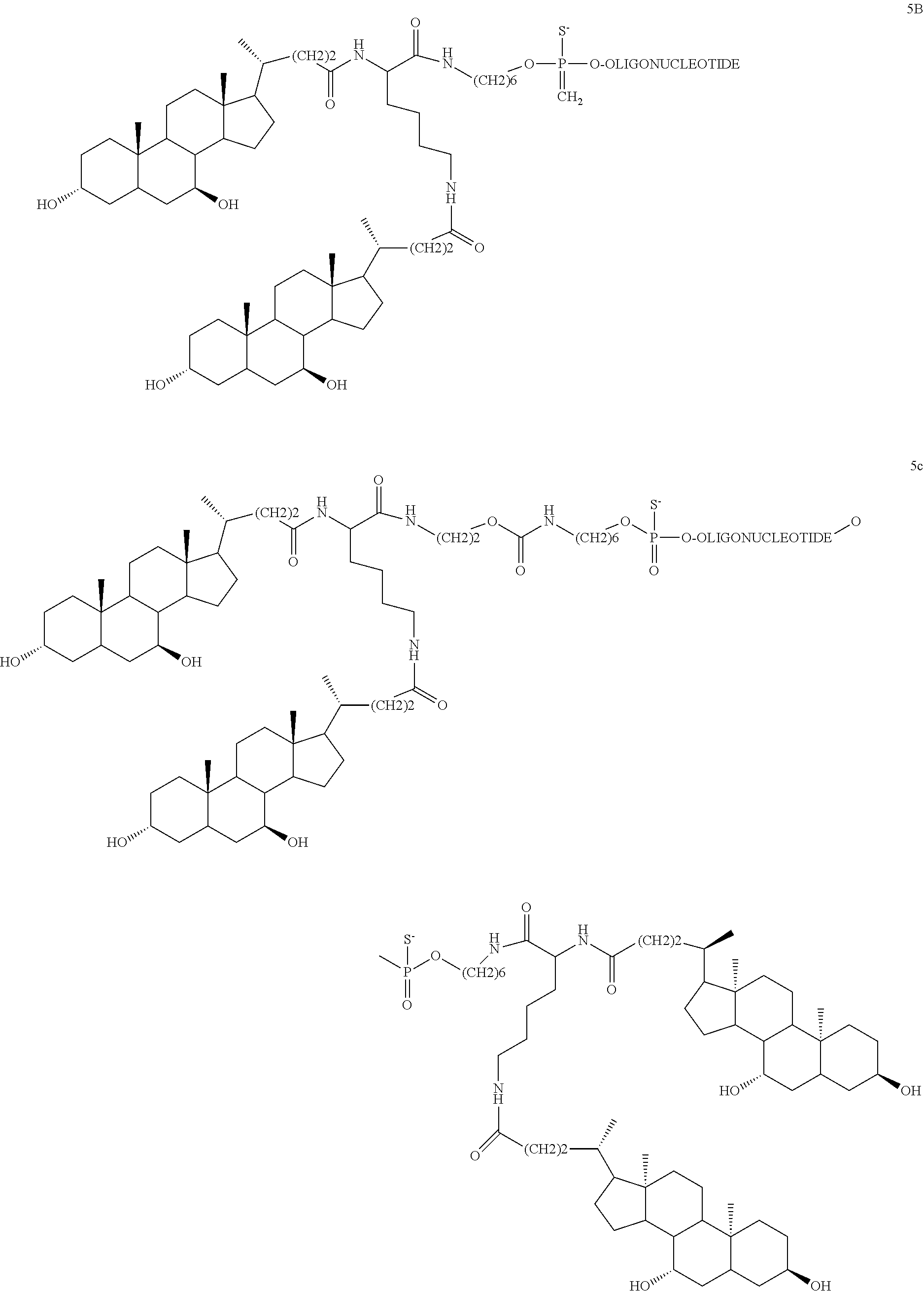

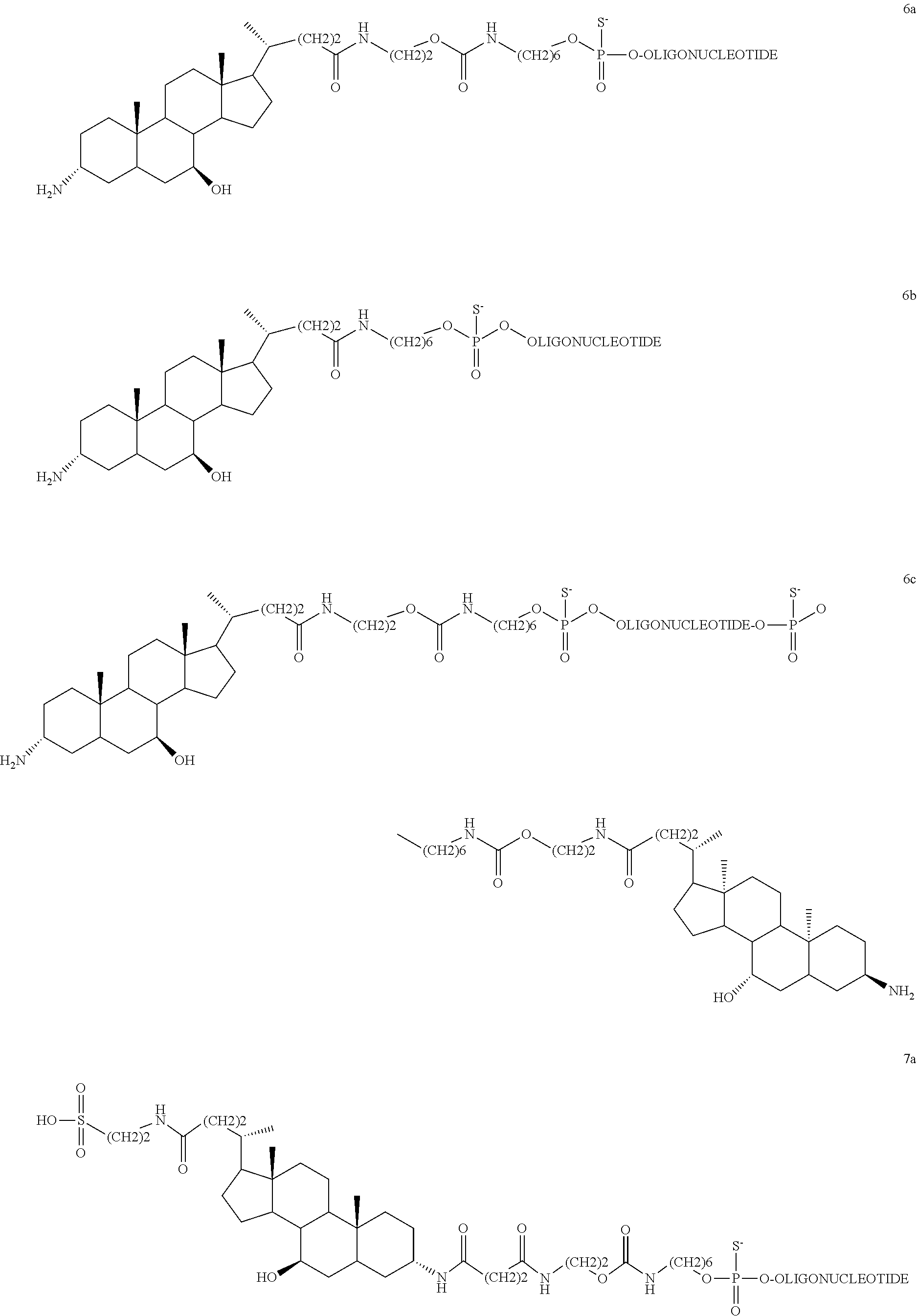

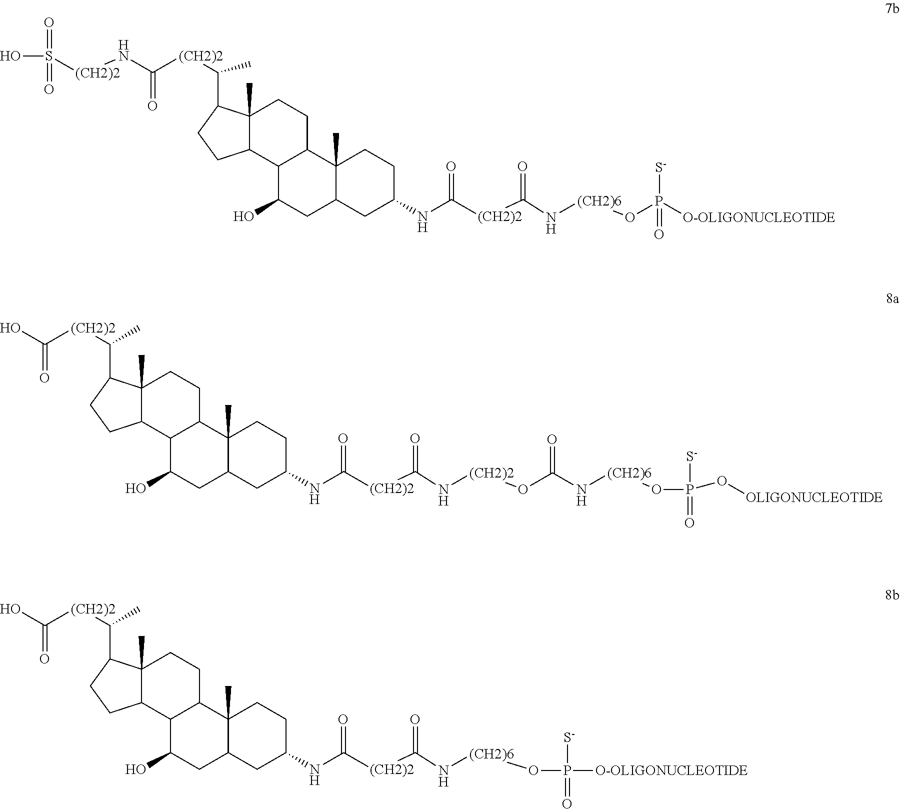

More preferably, the conjugates are selected from the group consisting of:

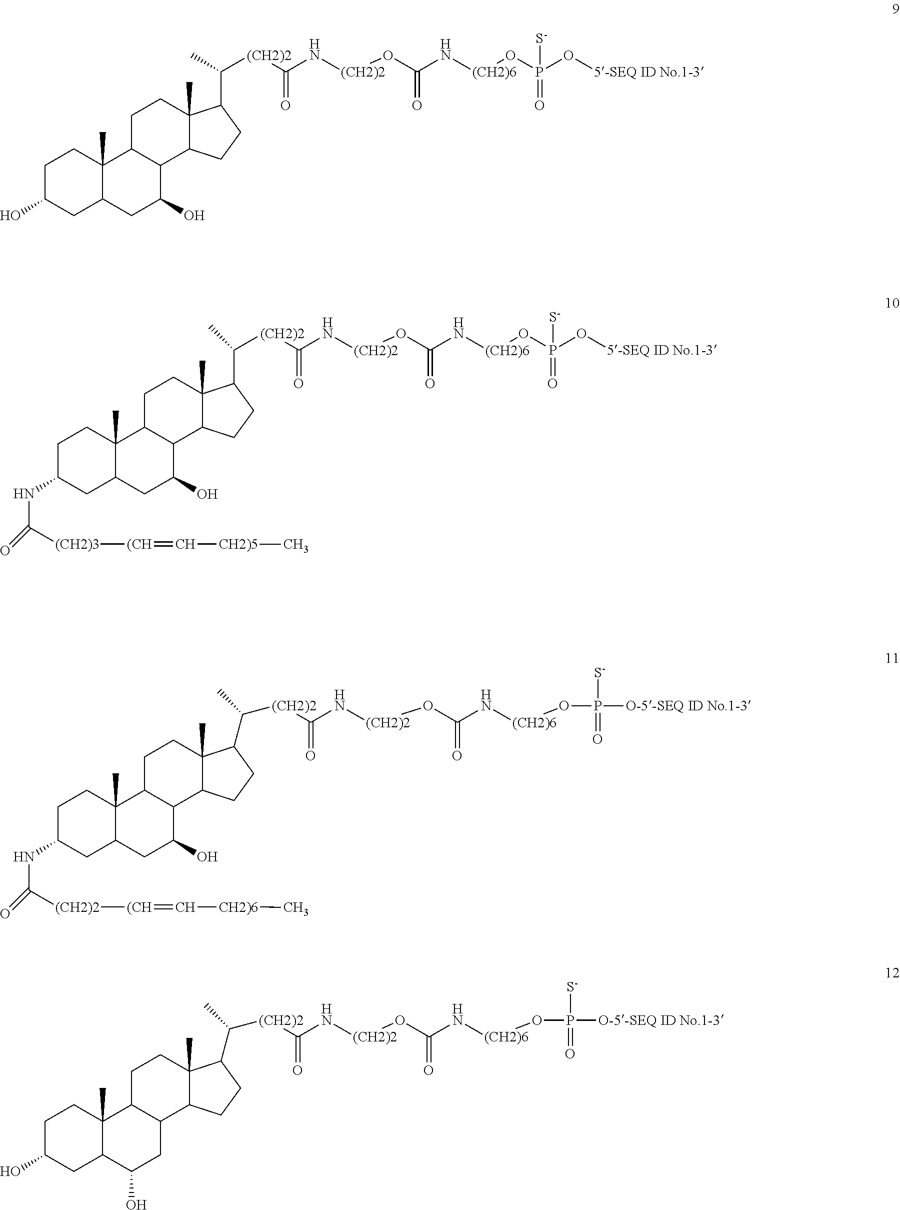

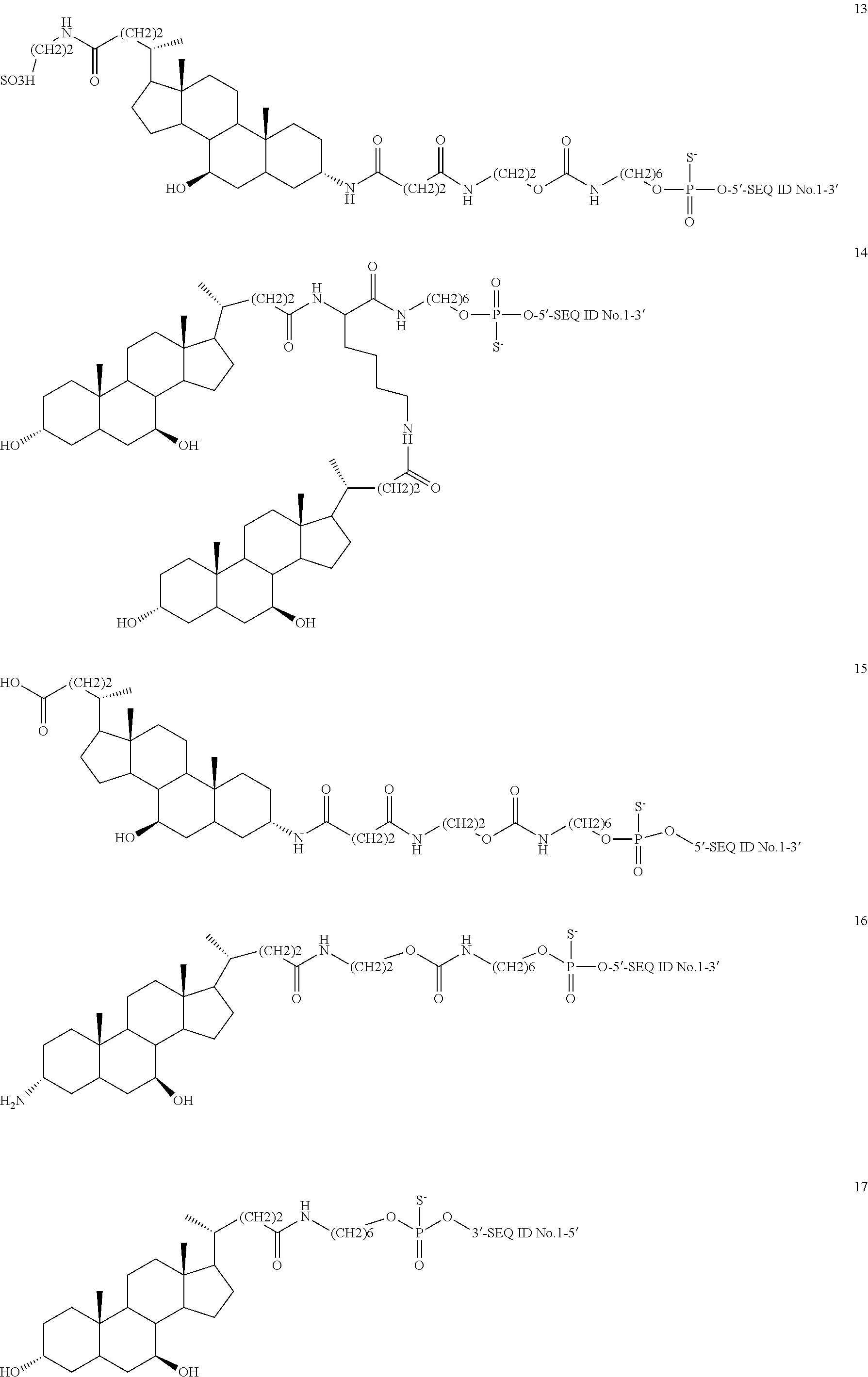

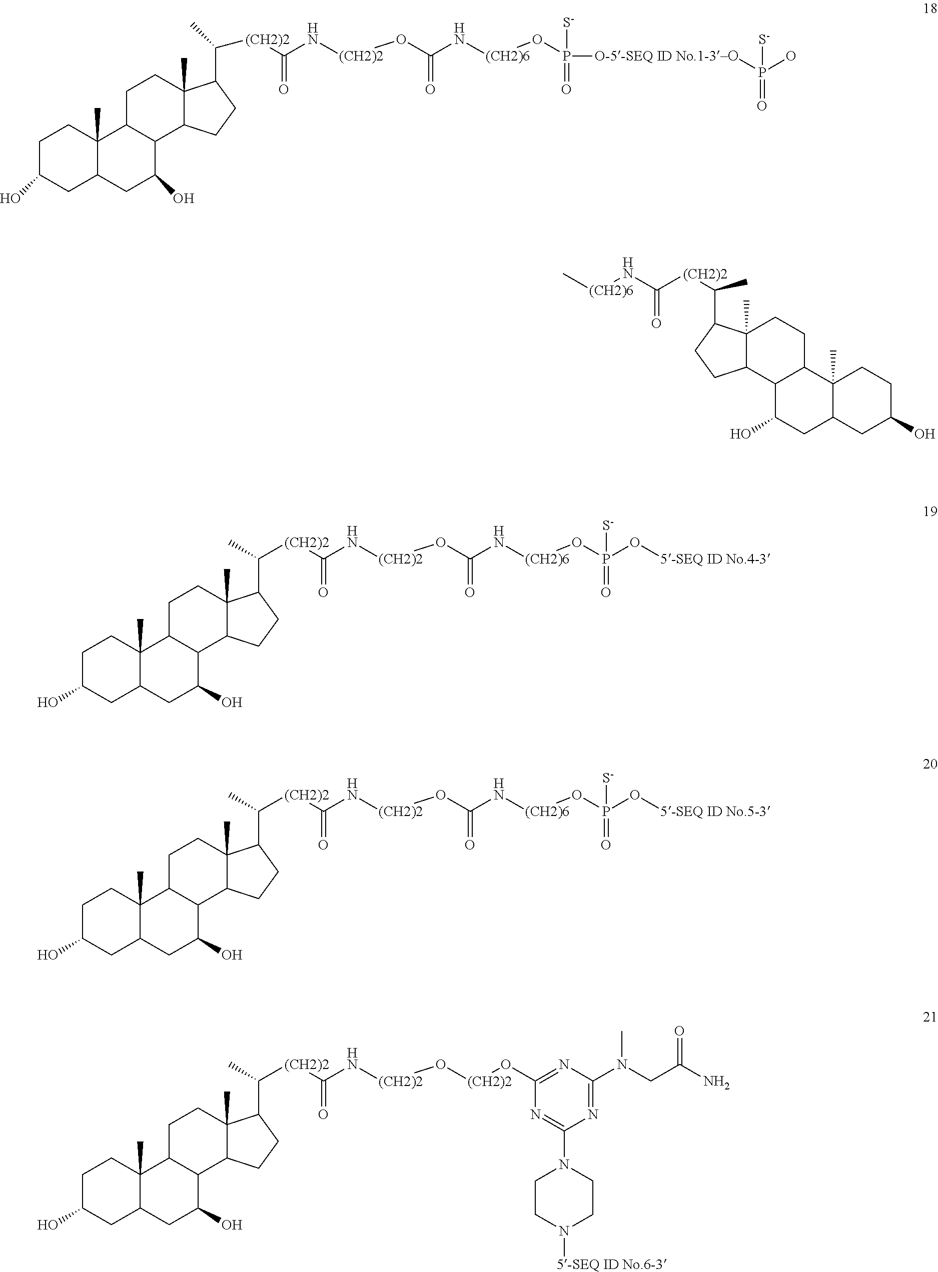

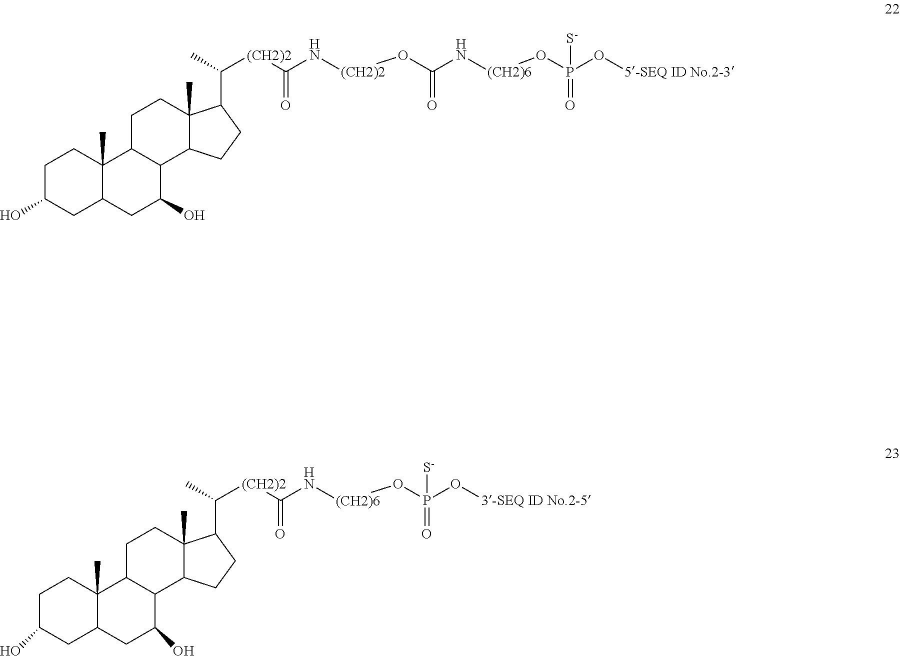

In accordance with a second aspect of the invention, pharmaceutical compositions of the compounds of Formula (I), (II) or (III) as described above are provided and at least one pharmaceutically acceptable excipient.

A person skilled in the art is familiar with an entire variety of said compounds and excipients suitable for formulating a pharmaceutical composition.

The compounds of the invention, together with a conventionally used excipient, can be in the form of pharmaceutical compositions and relative unit dosages, and in said form can be used as solids, as tablets or filled capsules, or liquids such as solutions, suspensions, emulsions, elixirs or capsules filled with the same, all for oral use or in the form of injectable sterile solutions for parenteral (including subcutaneous and intravenous) administration.

Said pharmaceutical compositions and relative unit dosage forms can comprise ingredients in conventional proportions, with or without additional compounds or active ingredients, and said unit dosage forms can contain any suitable effective quantity of the active ingredient in proportion to the scheduled daily dosage range to be used.

Pharmaceutical compositions containing a compound of this invention can be prepared in a way well-known in the pharmaceutical art and comprise at least one active compound. In general, the compounds of this invention are administered in a pharmaceutically effective quantity. The quantity of the compound actually administered will be typically determined by a doctor, in light of the relevant circumstances, including the condition to be treated, the chosen method of administration, the actual compound administered, the age, weight and response of the individual patient, the gravity of the patient's symptoms and the like.

The pharmaceutical compositions of the present invention can be administered by means of a variety of methods including oral, rectal, subcutaneous, intravenous, intramuscular, intranasal and pulmonary methods. The compositions for oral administration can take the form of liquid solutions or suspensions in mass, or powders in mass. More commonly, however, the compositions are presented in unit dosage forms to facilitate accurate dosage. The expression “unit dosage forms” refers to physically discrete units suitable as unit dosages for humans and other mammals, each unit containing a predefined quantity of active material calculated to produce the desired therapeutic effect in association with a suitable pharmaceutical excipient. Typical unit dosage forms include vials or pre-filled syringes, pre-measured with the liquid compositions or pills, tablets, capsules or similar in the case of solid compositions.

Liquid forms suitable for oral administration can include a suitable aqueous or non-aqueous carrier with buffer, suspension and dispersion agents, dyes, aromas and similar. Solid forms can include, for example, any one of the following ingredients, or compounds of a similar nature: a ligand such as microcrystalline cellulose, gum tragacanth or gelatin; an excipient such as starch or lactose, a disaggregating agent such as alginic acid, Primogel or maize starch; a lubricant such as magnesium stearate; a fluidifying agent such as colloidal silicon dioxide; a sweetening agent such as sucrose or saccharin or a flavouring agent such as peppermint, methyl salicylate or orange flavouring.

The injectable compositions are typically based on injectable sterile saline solution or saline solution buffered with phosphate or other injectable carriers known in the art.

The pharmaceutical compositions can be in the form of tablets, pills, capsules, solutions, suspensions, emulsions, powders, suppositories and as slow release formulations.

If desired, the tablets can be coated by means of standard aqueous or non-aqueous techniques. In some embodiments, said compositions or preparations can contain at least 0.1 percent of active compound. The percentage of active compound in these compositions can obviously be varied and can expediently be between approximately 1 percent and approximately 60% of the weight of the unit. The quantity of active compound in said therapeutically useful compositions is such that a therapeutically active dosage will be obtained. The active compounds can also be administered intranasally, such as, for example by liquid drops or spray.

The tablets, pills, capsules and similar can also contain a ligand such as gum tragacanth, acacia, maize starch or gelatin, excipients such as calcium phosphate, a disintegrating agent such as maize starch, potato starch, alginic acid, a lubricant such as magnesium stearate and a sweetening agent such as sucrose, lactose or saccharin. When a unit dosage form is a capsule, it can contain in addition to the materials of the above-mentioned type, a liquid carrier such as a fatty oil. Various other materials can be present as coatings or to modify the physical form of the dosage unit. For example, the tablets can be coated with shellac, sugar or both. A syrup or an elixir can contain, in addition to the active ingredient, sucrose as a sweetening agent, methyl and propyl parabens as preservatives, a dye and a flavouring agent such as cherry or orange flavour. To avoid disaggregation during transit through the upper portion of the gastrointestinal tract, the composition is a formulation with enteric coating.

The compositions for pulmonary administration include, but are not limited to, anhydrous powder compositions composed of the powder of a compound of Formula (I) or a relative salt and the powder of a carrier and/or suitable lubricant. The compositions for pulmonary administration can be inhaled from any anhydrous powder inhaler device known to a person skilled in the art.

The compositions are administered within the framework of a protocol and at a dosage sufficient to reduce the inflammation and the pain in the patient. In some embodiments, in the pharmaceutical compositions of the present invention, the active ingredient or the active ingredients are generally formulated in dosage units. The dosage unit can contain from 0.1 to 1000 mg of a compound of Formula (I) per dosage unit for daily administration.

In some embodiments, the effective quantities for a specific formulation will depend on the gravity of the disease, disorder or condition, the previous treatment, state of health of the individual and response to the drug. In some embodiments, the dose ranges from 0.001% by weight to approximately 60% by weight of the formulation.

When used in combination with one or more different active ingredients, the compound of the present invention and the other active ingredient can be used in lower doses than when each one is used individually.

As regards the formulations with respect to any variety of administration routes, methods and formulations for the administration of drugs are illustrated in Remington's Pharmaceutical Sciences, 17^{th} Edition, Gennaro et al. Eds, Mack Publishing Co., 1985 and Remington's Pharmaceutical Sciences, Gennaro AR ed. 20^{th} Edition, 2000, Williams & Wilkins PA, USA, and Remington: The Science and Practice of Pharmacy, 21^{st} Edition, Lippincott Williams & Wilkins Eds., 2005; and in Loyd V. Allen and Howard C. Ansel, Ansel's Pharmaceutical Dosage Forms and Drug Delivery Systems, 10^{th} Edition, Lippincott Williams & Wilkins Eds., 2014.

The components described above for compositions administered orally or injectable compositions are provided purely as examples.

The compounds of this invention can also be administered in slow release forms or by slow release drug delivery systems.

A third aspect of the present invention refers to compounds of Formula (I), (II) or (III) the pharmaceutical composition as illustrated above, for use as a medicament.

In particular, the conjugates of the invention can be used to improve the exon skipping in an mRNA of interest.

The conjugates of the invention can therefore be applied in the treatment of a disease selected from the group consisting of Duchenne dystrophy, Bardet-Biedel syndrome, beta thalassemia, cancer, cystic fibrosis, factor VII deficiency, familial dysautonomia, Fanconi anaemia, haemophilia A, propionic acidemia, retinitis pigmentosa, ataxia telangiectasia, congenital disorders of glycosylation, congenital adrenal insufficiency, Fukuyama congenital dystrophy, growth hormone insensitivity, BH4 deficiency hyperphenylalaninemia, Hutchinson-Gilford progeria, megalencephalic leukoencephalopathy with subcortical cysts, methylmalonic aciduria, myopathy with lactic acidosis, myotonic dystrophy, neurofibromatosis, Niemann-Pick disease type C, Usher syndrome, afibrinogenemia, ocular albinism type 1, Alzheimer's disease, tauopathies, spinal muscular atrophy, atherosclerosis, inflammatory diseases, muscular atrophy diseases, spinocerebellar ataxia type 1, dystrophic epidermolysis bullosa, and Miyoshi myopathy.

In particular, the conjugates having Formula (I), (I) and (III) in which the oligonucleotide is selected from those of SEQID No.1, SEQID No.2, SEQID No.3, SEQID No.4, SEQID No.5 and SEQID No.6 find particular application in the treatment of Duchenne dystrophy.

Further characteristics of the present invention will become clear from the following description of some merely illustrative non-limiting examples.

### EXAMPLES

### Example 1

### Method of Conjugation at the 5′ Terminal of the Oligonucleotide

### Synthesis of Conjugate 1a

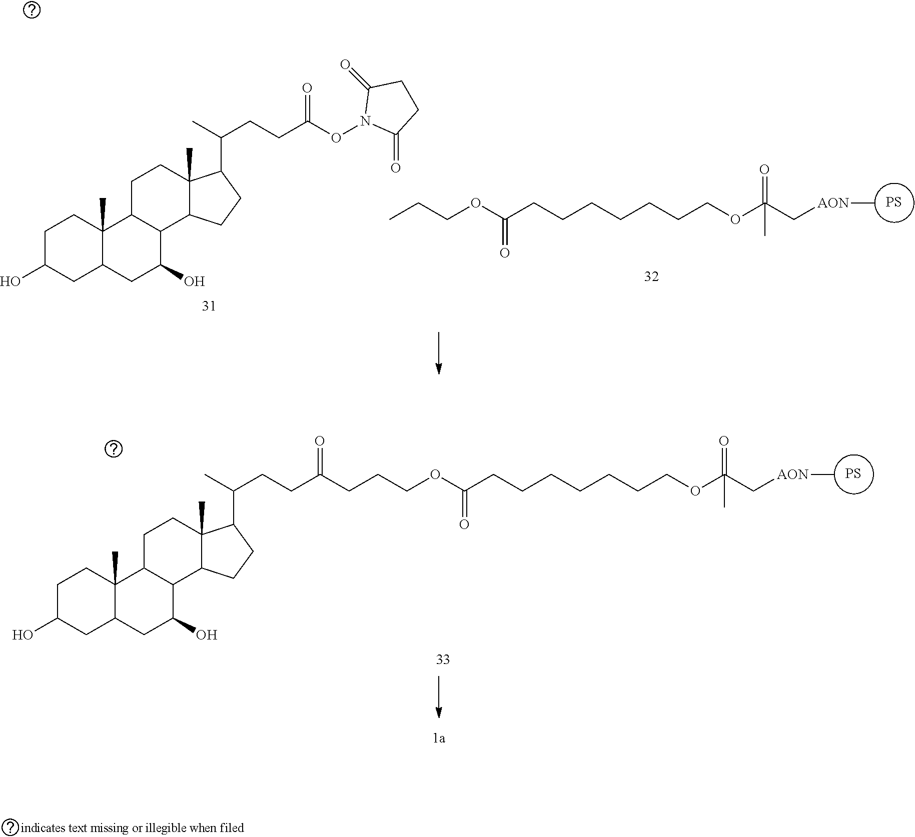

PS=polystyrene support

The conjugate 1a was prepared by reaction of the activated UDC 31 with the oligonucleotide 32 (AON=SEQ ID No. 1), functionalized in position 5′ with a commercial amine linker. The conjugation takes place in solid phase using a solution of the derivative 31 (10 eq) and DIPEA (40 eq) in a 1:1 mixture of DMSO/MeCN for 3 hours. Based on the synthesis scale, the flow is within the range 3-15 ml/min in recycle mode. The conjugate 1a was obtained following removal from the solid support and appropriate chromatographic purification in reverse phase using a gradient of buffer A (sodium acetate 0.1M+5% acetonitrile) and buffer B (acetonitrile). Lastly, the conjugate 1a was precipitated with ethanol from a 0.3M solution of sodium acetate. Conjugation efficiency ≥85%, overall process yield after purification 75%.

### Example 2

### Method of Conjugation at the 3′ Terminal of the Oligonucleotide

### Synthesis of the Conjugate 1b

### Example 2a: Conjugation in Solution

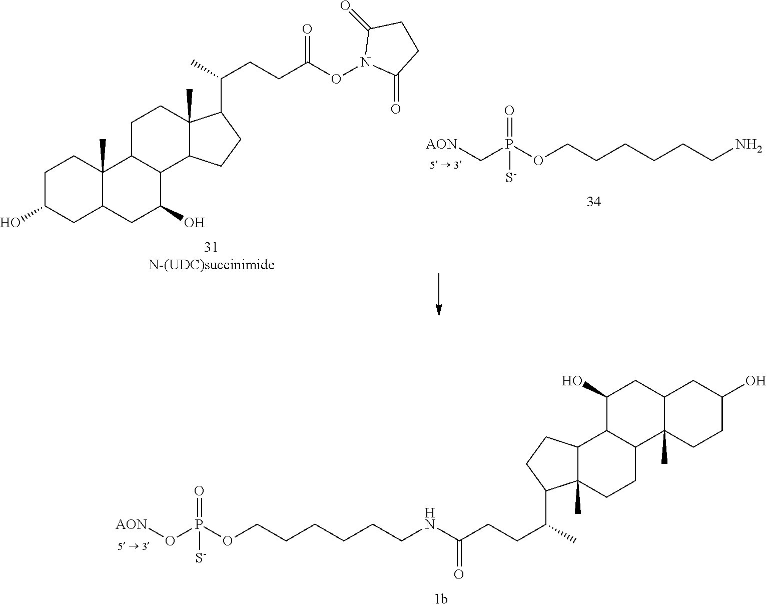

The oligonucleotide 34 (AON=SEQ ID No. 1) was synthesized in solid phase from the modified support C6 Amino Linker 300 (GE Primer Support™ 5G) in DMT-ON mode. Following removal from the solid support, purification in reverse phase and removal of the dimethoxytrityl group, the oligonucleotide 34 (5 mM solution in DMSO) was conjugated with the UDCA activated N-(UDC) 31 (2 eq) in the presence of DIPEA (40 eq). After 4 hours, the conjugated oligonucleotide 1b was further purified by means of reverse phase chromatography and precipitated from ethanol (conjugation efficiency ≥80%, overall process yield 60%).

### Example 2b: Conjugation in Solid Phase

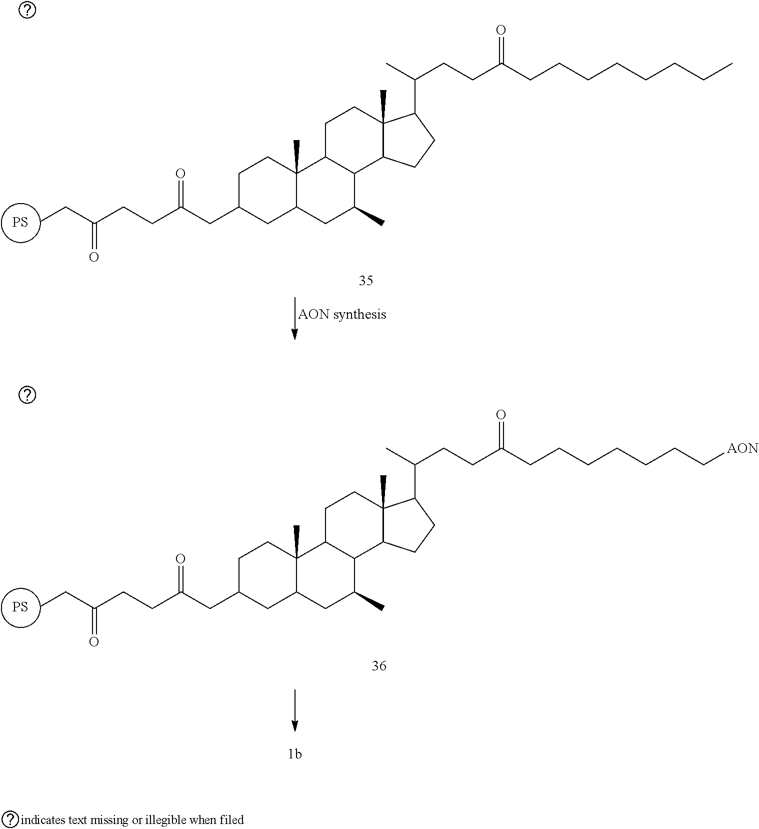

PS=polystyrene support

The conjugated oligonucleotide 36 was synthesized from the polystyrene support 35 (synthesis described below) in solid phase. After removal from the solid support and purification by means of reverse phase chromatography, the conjugated oligonucleotide 1b was obtained.

Synthesis of the polystyrene support 35

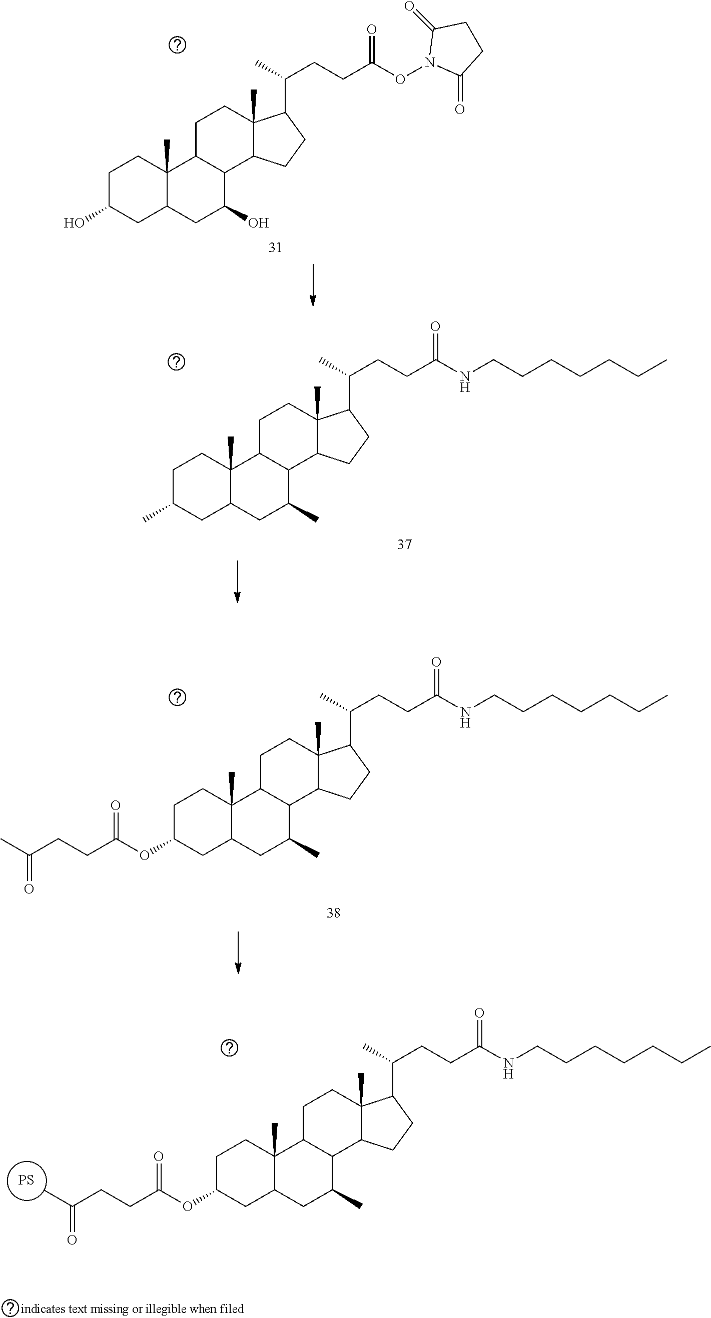

(i) 6-amino-1-hexanol, DIPEA, DMF; (ii) DMTrCl, pyridine, Ac₂O; (iii) KOH, EtOH; (iv) succinic anhydride, DMAP, TEA, DCM; (v) HBTU, DIPEA, polystyrene support, ACN, DMF.

Synthesis of the derivative 37: 6-amino-1-hexanol (1.2 g, 10.2 mmol) and DIPEA (0.6 ml, 4.1 mmol) were added to a solution of activated UDC 31 (1.0 g, 2.0 mmol) in 40 ml of anhydrous DMF. The reaction was kept under magnetic stirring for 22 hours at ambient temperature and then diluted with water and extracted with CH₂Cl₂. The organic phase was evaporated at reduced pressure and the residue obtained co-evaporated several times with toluene to give 1.0 g of raw compound used in the following step. The DMTr-Cl (0.50 g, 1.46 mmol) was added to a solution of the raw compound obtained previously (0.48 g, 0.98 mmol) and dissolved in anhydrous pyridine (5 mL). The reaction mixture was kept under stirring for 19 hours and then treated with 0.5 ml acetic anhydride for 3 h. 0.5 ml of ethanol were then added and the mixture was stirred for a further 30 minutes. The solvent was evaporated in the rotavapor and the raw reaction product obtained was re-dissolved in ethanol and treated with a 0.1M solution of KOH for 4 hours. The mixture was then neutralized by addition of a phosphate buffer, extracted with ethyl acetate and concentrated under a vacuum. Lastly, the raw product 37 was purified with flash chromatography (EtOAc:cyclohexane 4:1 with 0.3% Et₃N). Yield after 2 steps 25%. ¹H-NMR (400 MHz, CD₃OD, selected data) δ: 7.45-7.38 (m, 2H), 7.32-7.12 (m, 7H), 6.85-6.78 (m, 4H), 4.80-4.68 (m, 1H, H-7), 3.78 (s, 6H), 3.58-3.45 (m, 1H, H-3), 3.21-3.00 (m, 4H), 1.92 (s, 3H), 0.95 (d, 3H, J 6.44), 0.92 (s, 3H) , 0.71 (s, 3H). ESI-MS (ES+) m/z 858 (M+Na⁺).

Synthesis of UDC-hemisuccinate 38: the compound 37 (0.58 g, 0.70 mmol), the succinic anhydride (0.56 g, 5.69 mmol) and the DMAP (catal.) were dissolved in pyridine and caused to react under stirring at 70° C. until completion of the reaction. The solvent was evaporated and the residue re-dissolved in ethyl acetate was washed with a cold and diluted solution of acetic acid. 0.62 g (95%) of hemisuccinate 38 (purity estimated by means of ¹H-NMR≥95%) were obtained from the organic phase anhydrified on anhydrous Na₂SO₄ and evaporated at reduced pressure. ¹H-NMR (400 MHz, CD₃OD, selected data) δ: 7.42-7.40 (m, 2H), 7.42-7.15 (m, 7H), 6.85-6.80 (m, 4H), 4.98-4.80 (m, 2H, H-3 and −7), 3.76 (s, 6H), 3.22-3.00 (m, 4H), 2.60-2.50 (m, 4H), 1.92 (s, 3H), 0.96 (d, 3H, J 6.44), 0.94 (s, 3H), 0.69 (s 3H). ESI-MS (ES-) m/z 905 (M-H⁺).

Functionalization of an amine support with UDC-hemisuccinate (compound 35): the compound 38 (280 mg, 0.30 mmol) was dissolved in 5 ml of anhydrous MeCN (water content<10 ppm) and concentrated at reduced pressure (operation repeated at least twice to work in anhydrous conditions). The residue was dissolved in a 1:1 anhydrous mixture of MeCN and DMF and 0.1 ml of DIPEA was added. A commercial polystyrene support (0.70 g, amine content: 350 μmol/g) was added to the mixture and the suspension was gently stirred in an incubator at 25° C. for 15 minutes. HCTU (0.30 mmol) was then added and the stirring continued for 18 hours. The solution was then filtered and the support washed in the following order with CH₃CN×3, CH₂Cl₂×3, and then dried under a vacuum: 1 h at ambient temperature and then 18 h at 40° C. The support thus obtained was stirred again at 25° C. for 18 hours in the presence of a solution consisting of a mixture of the reagents CAP A and CAP B (Sigma-Aldrich; 5 ml of each solution). Lastly it was filtered again, washed and dried as previously described. The loading after the functionalization was measured equal to 240 μmol/g.

### Example 3

### Method of Conjugation at the 5′ and 3′ Terminals of the Oligonucleotide

### Synthesis of 5′-UDC-AON-UDC-3′ (1c)

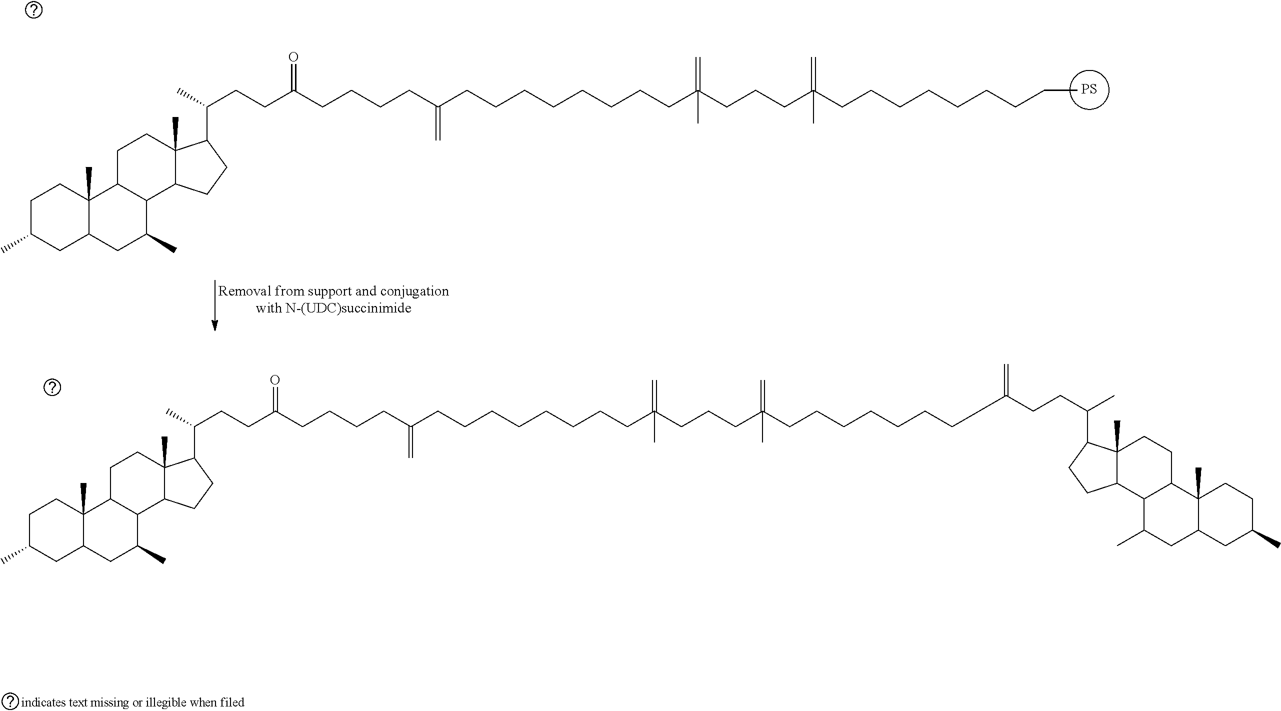

The synthesis of the oligonucleotide-bile acid conjugate 39 (AON=SEQ ID No. 1) was performed as reported previously for the conjugate 1a but starting from the modified solid support C6 Amino Linker 300 (GE Primer Support™ 5G). After removal from the solid support and purification in reverse phase, the resulting oligonucleotide (5 mM solution in DMSO) was caused to react with N-(UDC)succinimide 31 (2 eq) in the presence of DIPEA (40 eq). After 4 hours the conjugated oligonucleotide 1c was further purified by means of reverse phase chromatography and precipitated from ethanol. (Conjugation efficiency≥80%, overall process yield 50%).

### Example 4

### Synthesis of the Bile Acid Derivatives

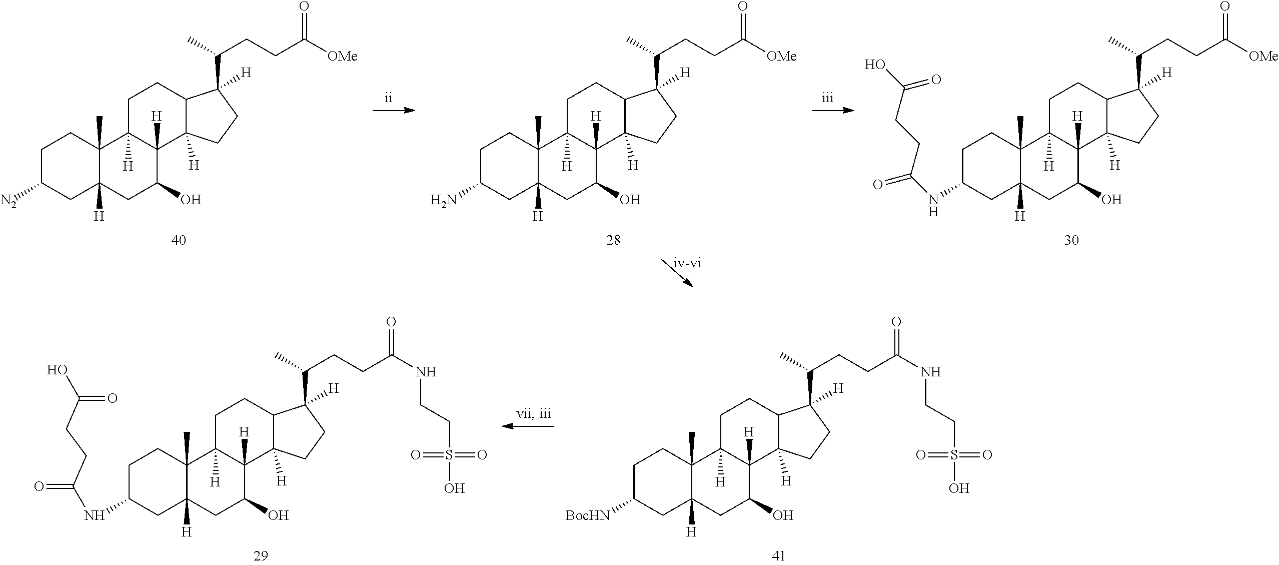

(ii) ammonium formate, Pd/C, AcOEt, MeOH; (iii) succinic anhydride, DMAP, pyridine, 115° C.; (iv) Boc₂O, NaHCO₃, THF, H₂O; (v) LiOH, H₂O, MeOH; (vi) ethyl chloroformate, TEA, taurine, NaOH, H₂O; (vii) TFA, CH₂Cl₂.

### Synthesis of 3α-NH₂-UDCA (28)

Pd/C (2.086 mmol) dissolved in MeOH (5 mL) was added slowly to a solution of 40 (1.043 mmol) and NH₄⁺HCOO⁻ (10.430 mmol) in AcOEt/MeOH 1:1 (10 mL). After 18 hours, during which the mixture was kept under stirring at 70° C., the Pd/C was separated by filtration. The solvent was evaporated at reduced pressure and the residue was extracted with CH₂Cl₂ (15 mL) and washed with brine (10 mL). The solution was anhydrified on anhydrous Na₂SO₄ and concentrated at reduced pressure to give the compound 28 as an amorphous white solid. Yield 78%. ¹H NMR (400 MHz, CDCl₃) : δ=3.62 (s, 3H, OMe), 3.59-3.50 (m, 1H, 7α-H), 2.64 (bs, 1H, 3β-H), 2.37-2.27 (m, 1H, 23-CH₂ₐ), 2.23-2.13 (m, 1H, 23-CH_{2b}), 1.99-0.85 (m, 30H), 0.64 (s, 3H, 18-CH₃). ¹³C NMR (101 MHz, CDCl₃): δ=174.66, 70.93, 55.83, 54.93, 51.45, 51.24, 43.73, 43.65, 42.89, 40.15, 39.23, 38.36, 37.19, 35.66, 35.32, 34.11, 31.10, 31.01, 28.62, 26.93, 23.63, 21.16, 18.35, 12.11. MS (ESI, ES+): Calculated for [C₂₅H₄₃NO₃+H]⁺ 406.63; found 406.33, 811.27 [2M+H]⁺, 1215.87 [3M+H]⁺. MS (ESI, ES-): calculated for [C₂₅H₄₃NO₃—H]⁻ 404.62; found 404.40, 805.07 [2M-H]⁻.

Synthesis of 3-hemisuccinyl-3α-amino-UDCMe (30)

Succinic anhydride (3.390 mmol) and DMAP in catalytic quantity were added to a solution of the amine derivative 28 (0.678 mmol) in pyridine (4 mL). The mixture was kept at 115° C. for 18 hours, then cooled at ambient temperature, diluted with AcOEt (15 mL) and washed with an aqueous solution of HCl at 5% (3.5 mL) and H₂O (5 mL). The extract was anhydrified on Na₂SO₄ and the solvent evaporated at reduced pressure to give the amorphous white solid 30. Yield 70%. ¹H-NMR (400 MHz, CDCl₃): δ=5.66 (d, J=7.9 Hz, 1H, NH), 3.77-3.67 (m, 1H, 7α-H), 3.65 (s, 3H, OMe), 3.57-3.41 (m, 1H, 3β-H), 2.96 (t, J=7.1 Hz, 2H, succinic CH₂), 2.53 (t, J=7.1 Hz, 2H, succinic CH₂), 2.40-2.29 (m, 1H, 23-CH₂ₐ), 2.26-2.16 (m, 1H, 23-CH_{2b}), 2.07-0.99 (m, H), 0.94 (s, 3H, 19-CH₃), 0.91 (d, J=6.4 Hz, 3H, 21-CH₃), 0.66 (s, 3H, 18-CH₃). ¹³C NMR (101 MHz, CDCl₃) : δ=174.72, 169.11, 168.98, 168.16, 71.37, 55.84, 55.03, 51.51, 50.43, 49.47, 43.71, 42.74, 40.17, 39.26, 36.71, 35.38, 35.28, 34.30, 34.04, 32.88, 31.09, 31.04, 28.60, 27.52, 27.04, 26.88, 25.57, 25.18, 24.49, 23.52, 21.14, 18.37, 12.11.

Synthesis of 3-hemisuccinyl-3α-amino-TUDCA (29)

Boc₂O (2.4 mmol) was added to a solution of the compound 28 (1.19 mmol) dissolved in THF (5 mL) and NaHCO₃ (saturated solution, 5 mL) and the solution was left under magnetic stirring for 18 hours. The residue was diluted with water and extracted with AcOEt (2×10 mL). The organic phase was anhydrified with anhydrous sodium sulphate, filtered and evaporated at reduced pressure. The solid thus obtained was re-dissolved in an aqueous solution of LiOH and MeOH. After 48 hours under stirring, the methanol was removed and the solution brought to pH 3 adding an aqueous solution of HCl 5%. The solution obtained was extracted with EtOAc (2×10 mL) and the re-combined organic phases, anhydrified with Na₂SO₄, were filtered and concentrated at reduced pressure. The raw residue thus obtained was not isolated but dissolved directly in THF (5 mL) and caused to react at 0° C. with triethylamine (1.3 mmol) and ethyl chloroformate (1.3 mmol). After 2 hours at ambient temperature, a solution of taurine (1.3 mmol) in NaOH/H₂O (1.43 mmol) was added. The reaction was kept under stirring at ambient temperature for 12 hours and then acidified to pH1 with HCl 5%. The THF was then evaporated under vacuum and the mixture dissolved in water was extracted with EtOAc. The aqueous phase was then extracted with n-butanol which, concentrated at reduced pressure, resulted in an amorphous white solid 41. The latter dissolved in dichloromethane was caused to react with TFA until complete removal of the Boc (24 hours). The mixture was then concentrated at reduced pressure and the solid obtained caused to react with succinic anhydride as described previously for the compound 30 to give the solid 29 (yield 5 steps 15%).

MS (ESI, ES-): calculated for [C₃₀H₅₀N₂O₈S—H]⁻ 597.80; found 597.47.

### Synthesis of the Derivative Lysine Bis-ursodeoxycholic Amide (27)

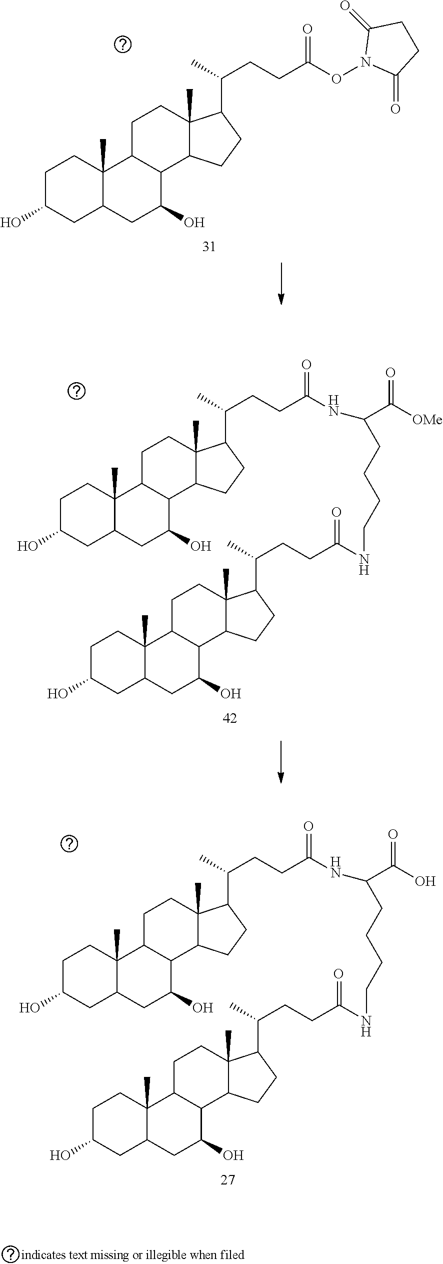 [SMILES: C[C@H](CCC(=O)ON1C(=O)CCC1=O)C1CCC2C3C(CC[C@@]21C)[C@@]1(C)CC[C@@H](O)CC1C[C@@H]3O ; COC(=O)C(CCCCNC(=O)CC[C@@H](C)C1CCC2C3C(CC[C@@]21C)[C@@]1(C)CC[C@@H](O)CC1C[C@@H]3O)NC(=O)CC[C@@H](C)C1CCC2C3C(CC[C@@]21C)[C@@]1(C)CC[C@@H](O)CC1C[C@@H]3O ; C[C@H](CCC(=O)NCCCCC(NC(=O)CC[C@@H](C)C1CCC2C3C(CC[C@@]21C)[C@@]1(C)CC[C@@H](O)CC1C[C@@H]3O)C(=O)O)C1CCC2C3C(CC[C@@]21C)[C@@]1(C)CC[C@@H](O)CC1C[C@@H]3O]

(i) lysine OMe, DIPEA; (ii) NaOH, H₂O, MeOH L-Lysine methyl ester dihydrochloride (0.899 mmol) and DIPEA (4.494 mmol; 785 μL) were added, at 0° C., to a solution of 31 (2.247 mmol) in CH₂Cl₂ (20 mL). The mixture was kept under stirring at 25° C. for 18 hours, after which 10 ml of aqueous solution of HCl 5% were added. The white solid 42 that precipitates from the reaction environment was filtered, redissolved in methanol and used in the following step without further purification. Yield 95%.¹H-NMR (400 MHz, DMSO-d₆): δ=8.14 (d, J=7.5 Hz, 1H, NHCH), 7.78 (t, J=5.6 Hz, 1H, NHCH₂), 4.47 (t, J=3.8 Hz, 2H, 2OH), 4.19-4.09 (m, 1H, NHCH), 3.88 (d, J=6.8 Hz, 1H, OH), 3.60 (s, 3H, OMe), 3.33-3.22 (m, 4H, 3β-, 3′β-, 7α- and 7′α-H di UDC), 3.16 (d, J=4.7 Hz, 1H, OH), 3.05-2.94 (m, 2H, NHCH₂), 2.28-0.84 (m, 68H), 0.61 (d, J=3.2 Hz, 6H, 18- and 18′-CH₃ di UDC). ¹³C-NMR (101 MHz, DMSO-d₆) δ=172.77, 172.30, 69.58, 69.33, 55.76, 54.60, 51.69, 51.56, 48.47, 42.95, 42.88, 42.05, 38.61, 38.11, 37.77, 37.60, 37.15, 34.86, 34.71, 33.64, 32.36, 31.92, 31.58, 31.43, 30.28, 30.13, 28.56, 28.08, 26.61, 24.77, 23.21, 22.62, 20.74, 18.36, 11.91. MS (ESI, ES+): calculated for [C₅₅H₉₂N₂O₈+H]⁺ 910.36; found 909.53, 1818.87 [2M+H]⁺; calculated for [C₅₅H₉₂N₂O₈+Na]⁺ 931.33; found 931.80, 1840.93 [2M+Na]⁺. MS (ESI, ES-): calculated for [C₅₅H₉₂N₂O₈+Cl]⁻ 943.79; found 943.53.

NH₄OH (7 mL) was added to a solution of 42 (0.649 mmol) in MeOH (7 mL), and the mixture was kept under stirring at 60° C. for 36 hours. The solvent was then removed at reduced pressure, an aqueous solution at 5% of HCl was added and the solid precipitated was filtered on Büchner and dried in a stove at 80° C. for 24 hours. The amorphous white solid 27 was obtained with a yield of 79%. ¹H-NMR (400 MHz, DMSO-d6): δ=7.94 (d, J=7.4 Hz, 1H, NHCH), 7.75 (t, J=8.2 Hz, 1H, NHCH₂), 7.27 (s, 1H), 6.93 (s, 1H), 4.50 (s, 2H, 2OH), 4.19-4.03 (m, 1H, NHCH), 3.90 (d, J=6.3 Hz, 1H, OH), 3.41-3.21 (m, 4H, 3β-, 3′β-, 7α- and 7′α-H di UDC), 3.16 (s, 1H, OH), 2.98 (bs, 2H, NHCH₂), 2.24-0.80 (m, 68H), 0.60 (d, J=1.1 Hz, 6H, 18- and 18′-CH₃ di UDC). ¹³C-NMR (101 MHz, DMSO-d₆) δ=173.96, 173.85, 172.56, 172.42, 69.66, 69.40, 55.82, 54.64, 52.07, 51.68, 43.02, 42.93, 42.10, 38.69, 38.05, 37.95, 37.64, 37.18, 35.06, 34.94, 34.75, 33.69, 32.42, 32.26, 32.13, 31.63, 31.54, 30.60, 30.16, 28.75, 28.66, 28.15, 26.66, 23.26, 22.76, 22.71, 20.80, 18.43, 11.97. MS (ESI, ES+): calculated for [C₅₄H₉₀N₂O₈+H]⁺ 896.33; found 895.47, 1789.80 [2M+H]⁺; calculated for [C₅₄H₉₀N₂O₈+Na]⁺ 917.30; found 917.67, 1811.87 [2M+Na]⁺. MS (ESI, ES-): calculated for [C₅₄H₉₀N₂O₈—H]⁻ 894.31; found 893.67, 1788.73 [2M-H]⁻.

### Example 5

### Synthesis of the Bile Acid-Fatty Acid Conjugates 24 and 25

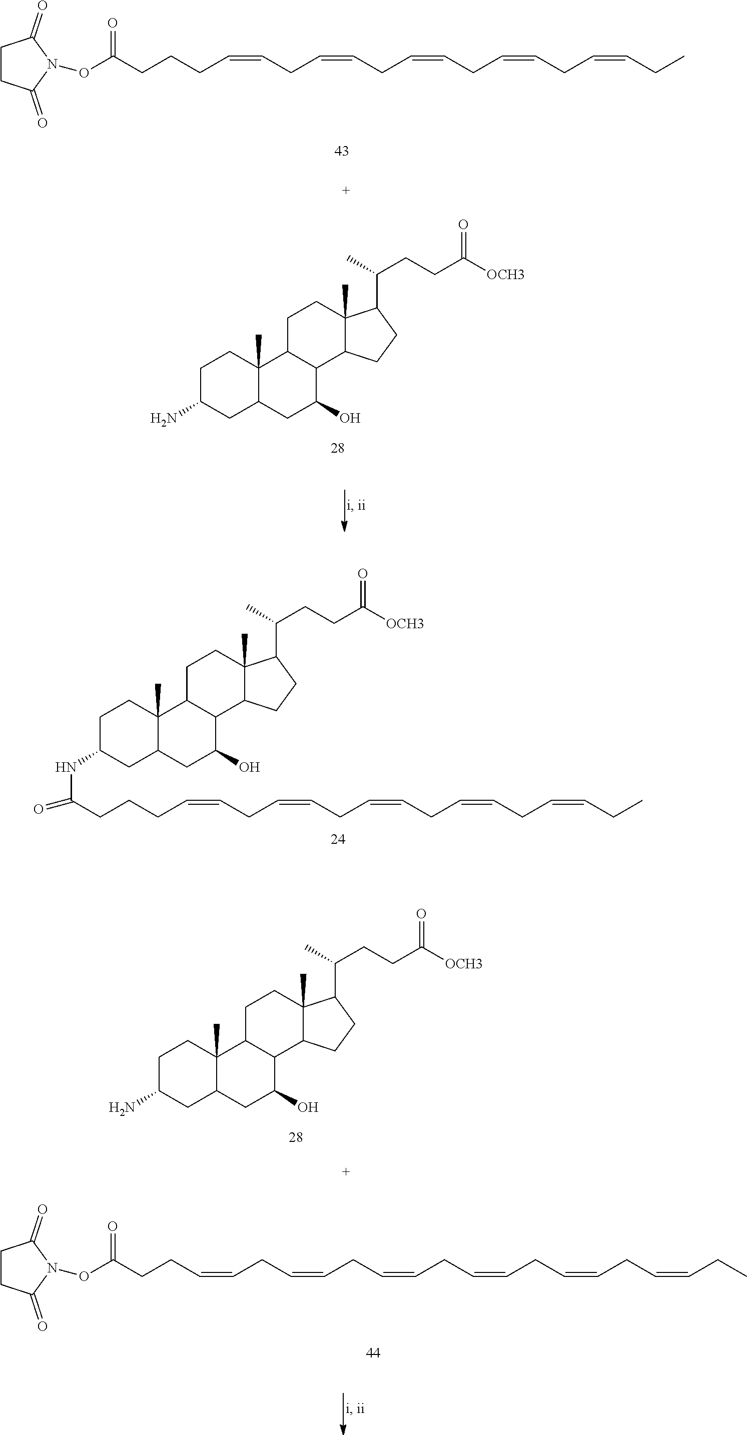 [SMILES: CC/C=C\C/C=C\C/C=C\C/C=C\C/C=C\CCCC(=O)ON1C(=O)CCC1=O ; COC(=O)CC[C@@H](C)C1CCC2C3C(CC[C@@]21C)[C@@]1(C)CC[C@@H](N)CC1C[C@@H]3O ; CC/C=C\C/C=C\C/C=C\C/C=C\C/C=C\CCCC(=O)N[C@@H]1CC[C@@]2(C)C(C1)C[C@H](O)C1C2CC[C@@]2(C)C1CCC2[C@H](C)CCC(=O)OC ; COC(=O)CC[C@@H](C)C1CCC2C3C(CC[C@@]21C)[C@@]1(C)CC[C@@H](N)CC1C[C@@H]3O ; CC/C=C\C/C=C\C/C=C\C/C=C\C/C=C\C/C=C\CCC(=O)ON1C(=O)CCC1=O]

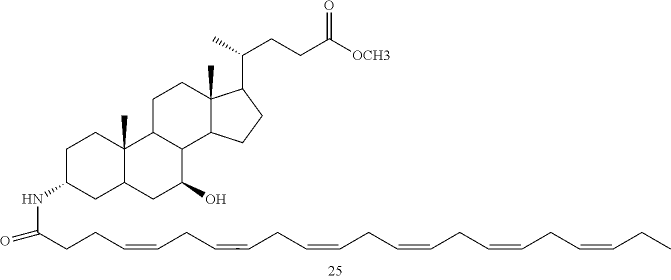 [SMILES: CC/C=C\C/C=C\C/C=C\C/C=C\C/C(C)=C\C/C=C\CCC(=O)N[C@@H]1CC[C@@]2(C)C(C1)C[C@H](O)C1C2CC[C@@]2(C)C1CCC2[C@H](C)CCC(=O)OC]

(i) DIPEA, DMF; (ii) NaOH, H₂O, MeOH

Synthesis of DH-UDC (25)

The amino-UDC 28 (1.752 mmol) and DIPEA (3.504 mmol; 491 μL) were added to a solution of 43 (1.752 mmol) in DMF (10 mL). After 18 hours of stirring at 25° C., 10 ml of HCl 5% was added to the mixture which was then extracted with CH₂Cl₂ (30 mL). The organic phase was further washed with NaHCO₃ (3·10 mL), then anhydrified with sodium sulphate and concentrated under vacuum. The flash chromatography (AcOEt/cyclohexane 1:1) resulted in isolation of an amorphous yellow solid with a yield of 49%. ¹H NMR (400 MHz, CDCl₃): δ=5.46-5.23 (m, 12H), 3.71 (bs, 1H, 3β-H), 3.66 (s, 3H, OMe), 3.57-3.48 (m, 1H, 7α-H), 2.89-2.73 (m, 10H, ═CH—CH₂—CH═), 2.45-2.29 (m, 3H), 2.26-2.14 (m, 3H), 2.12-0.83 (m, 35H), 0.67 (s, 3H, 18-CH₃). ¹³C NMR (101 MHz, CDCl₃): δ=174.72, 171.43, 132.04, 129.23, 128.56, 128.26, 128.06, 127.84, 126.97, 71.34, 55.84, 54.99, 51.54, 49.14, 43.73, 42.76, 40.15, 39.28, 36.66, 35.43, 35.26, 34.63, 34.05, 31.06, 31.01, 28.60, 27.81, 26.87, 25.63, 25.54, 24.89, 23.55, 21.13, 20.56, 18.37, 14.29, 12.11. MS (ESI, ES+) : calculated for [2.C₄₇H₇₃NO₄+3H]³⁺ 478.41; found 479.50; calculated for [2.C₄₇H₇₃NO₄+3Na]³⁺500.39; found 503.37. The solid obtained was then hydrolysed to give the acid 25 as described for the compound 27.

### Example 6

In Vitro Study of the Skipping Efficiency of the Conjugates of the Invention

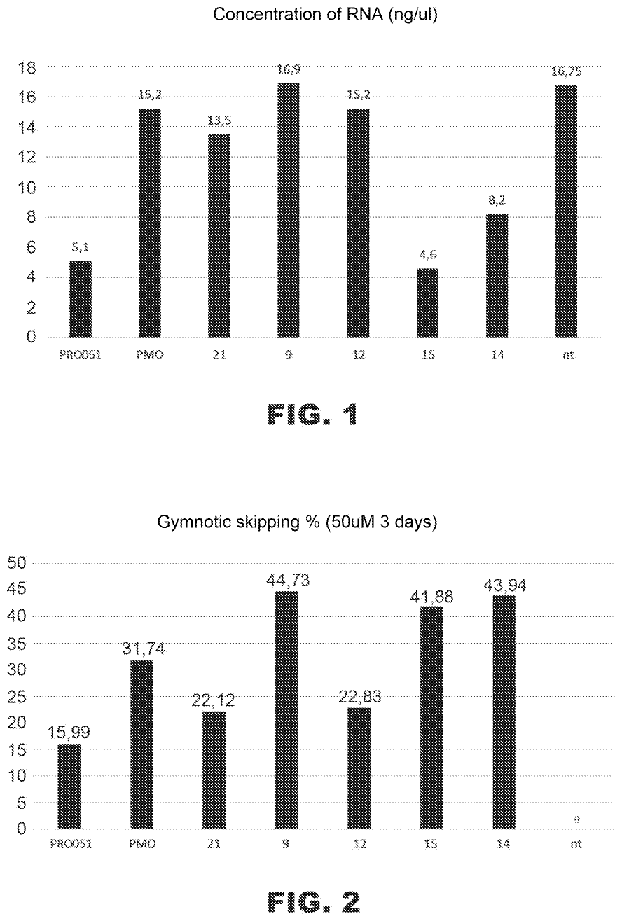

The myotubes obtained by differentiation of a cell line of immortalized human myoblasts derived from a patient with deletion of the exon 52 of the dystrophin gene (DMD) were treated with a concentrated solution of each of the molecules indicated in FIG. 1 (PRO051=SEQ ID No. 1, PMO=SEQ ID No. 6, compound 21, compound 9, compound 12, compound 15, compound 14 and nt=not treated). The treatment was conducted in the absence of transfectant in plates with 48 wells, to obtain a final concentration of 50 μM in each well. After 72 hours, the cells were collected for extraction of the RNA, the concentrations of which are reported in FIG. 1.

200 ng of each RNA were retrotranscribed by successive amplification with primers complementary to exon 50 (Ex50F) and 54 (Ex54R) for 28 cycles of RT-PCR.

One microlitre of the RT-PCR was then analysed by means of Bioanalyser 2100 Agilent to quantify the products relative to the deleted transcript of the exon 52 (control) and the transcript resulting from the skipping of the exon 51 induced by the antisense oligonucleotides.

The skipping efficiency for each compound is reported in FIG. 2.

All the conjugates of the antisense oligonucleotide reported in the graph of FIG. 2 show a greater skipping effectiveness than PRO051 (SEQ ID No. 1). In particular, all the compounds in which the SEQ ID No. 1 is conjugated with UDCA (compounds 9, 14) or its 3-amino derivative (compound 15) show a skipping efficiency greater than 40%.

In Vitro and In Vivo Study of the Effectiveness of New Antisense Oligonucleotides Conjugated with UDCA

Myotubes obtained by differentiation of a cell line of immortalized human myoblasts derived from a patient with deletion of the exon 52 of the dystrophin gene (DMD), which interrupts the reading frame, were treated with 2 μl of a 100 μM solution of the following conjugates of the antisense oligonucleotide of SEQ ID No.1 complementary to the exon 51 of the DMD gene: compounds 9, 17 and PRO051 (SEQ ID No. 1).

The treatment was conducted with Turbofect transfectant in plates with 24 wells and the cells collected 48 hours or five days after for extraction of the RNA or immunofluorescence analysis respectively.

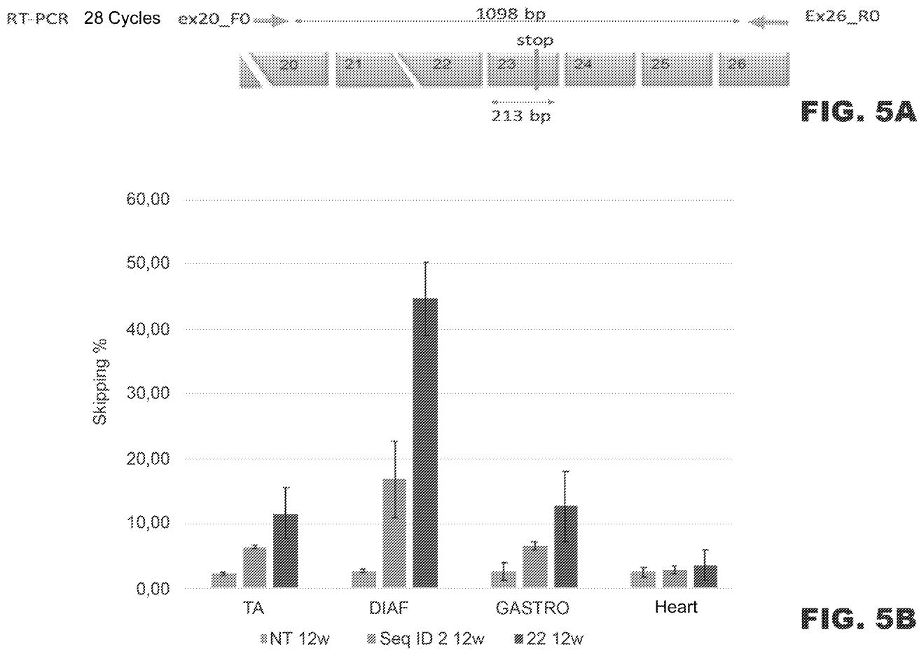

The RNA was quantified and retrotranscribed by successive amplification with primers complementary to the exon 50 (Ex50F) and 54 (Ex54R) for 28 cycles of RTPCR (FIG. 5A). One microlitre of the RT-PCR was subsequently analysed by means of Bioanalyser 2100 Agilent to quantify the products relative to the deleted transcript of the exon 52 (control) and the transcript resulting from skipping of the exon 51 induced by the antisense oligonucleotides.

For the immunofluorescence analysis, the myoblasts were treated for five days with 4 μl of a 100 μM solution of each of the antisense oligonucleotides in appropriate plates, before being fixed and labelled with antibodies NCL-DYS2 and DAPI.

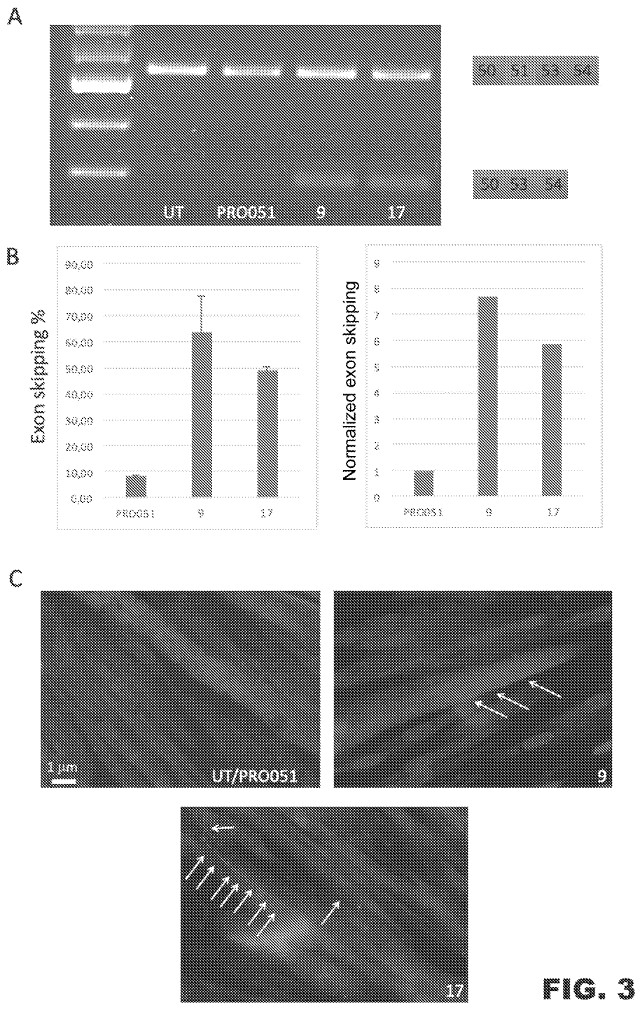

From analysis of the dystrophin transcript, it was found that the conjugated antisense oligonucleotides (compounds 9 and 17) are both more effective than the PRO051 in inducing exon skipping (FIG. 3A). In fact, by setting to one the quantity of naturally missing transcript of the exon 51 with respect to the total of the dystrophin transcript in the non-treated cells, it was possible to quantify the skipping in the cells treated with PRO051 (8.33±0.53%), with antisense conjugated at 5′ (compound 9) (63.75±13.77%) and with antisense conjugated at 3′ (compound 17) (49±1.41%) (FIG. 3B, histograms on the left).

Therefore, the antisense conjugates induced a skipping increment of 7.65 times (compound 9) and 5.88 times (compound 17) compared to the oligonucleotide PRO051.

The immunofluorescence analysis showed restored expression of the dystrophin and correct localization at the sarcolemma only in the myotubes treated with the conjugated antisense oligonucleotides (compounds 9 and 17) (FIG. 3C).

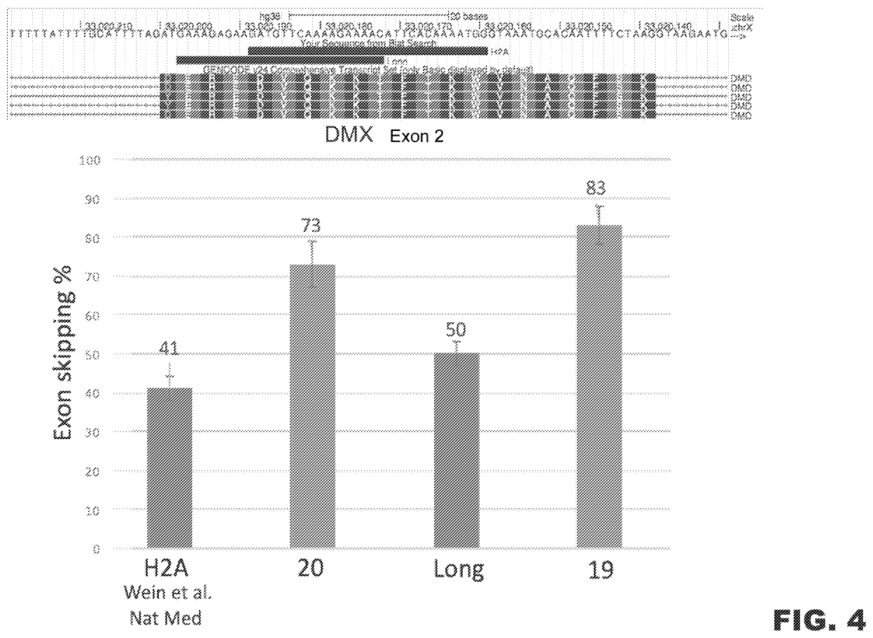

The following conjugates of antisense oligonucleotides directed against the exon 2 of the dystrophin gene (FIG. 4) were also tested in a control cell line: compounds 19, 20, SEQ ID No.4 (indicated in FIG. 4, as Long) and SEQ ID No.5 (indicated in FIG. 4, as H2A).

Analysis of the transcript showed skipping of the exon 2 in a percentage of 41% for the oligonucleotide SEQ ID No.5 and 73% for the compound 20; 50% for the oligonucleotide SEQ ID No.4 and 83% for the compound 19 (FIG. 4, histograms).

To test in vivo the effectiveness of the antisense oligonucleotides conjugated with UDCA, the oligonucleotides 22 and SEQ ID No.2 were injected intraperitoneally at the dose of 200 mg/kg with regime of one administration per week for 12 weeks in male mice C57BL/10ScSn-Dmdmdx/J of 2 months. Mice injected with PBS were used as controls.

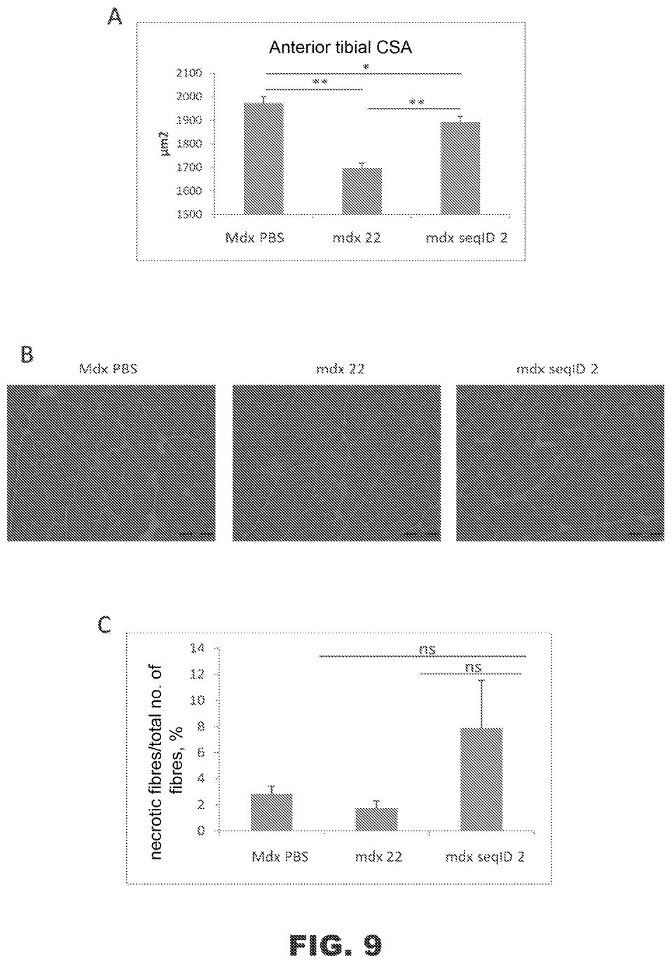

During the treatment, the mice were constantly monitored for any symptoms of pain or unwellness and their body weight was recorded twice a week. During the experiment the mice did not show any signs of unwellness or illness. On the contrary, they were vital, as emerges from FIG. 9 which reports their body weight trend recorded during the experiment.

Motor coordination and neuromuscular strength were monitored with the four limb hanging test, in accordance with the TreatNMD DMD_M.2.1.005 guidelines. Briefly, the protocol establishes that the mouse is placed on a grille which is overturned, recording the time (in seconds) that elapses until the animal falls off. This test was performed at different times during the experiment and two times were recorded for each mouse, one an hour after the other. Lastly, each time was normalized for the body weight and the longest of the two was taken into account (Maximum holding impulse, g*s). The mice treated with the compound 22 showed an interesting improvement trend in neuromuscular strength from the beginning of the treatment through to the eighth week, with respect to the other experimental conditions (FIG. 8).

In the week following the last treatment, the mice were sacrificed to collect samples of heart, diaphragm, gastrocnemius muscle and anterior tibial muscle. The muscles were divided and fragmented to conduct the histological analyses, the immunofluorescence analyses, the quantifications by means of LC/MS/MS, analysis of the transcript and quantification of the proteins.

### Histological Analyses

The histological analysis performed on the anterior tibial, gastrocnemius and diaphragm muscles gave different results.

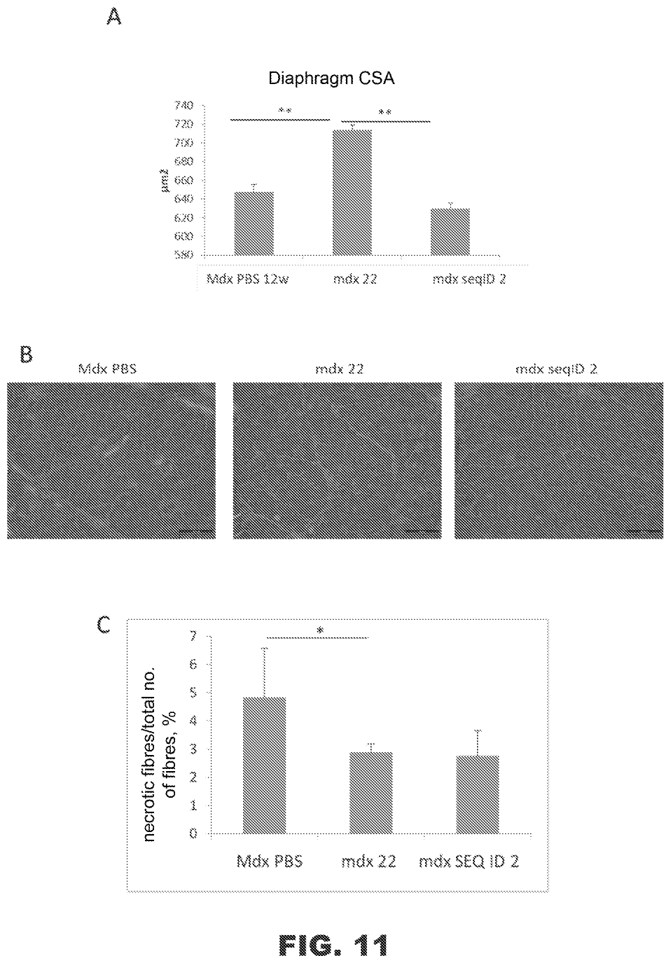

The analysis performed on the anterior tibial muscle highlighted a reduction in the mean cross-section area (CSA) of the fibres in the mdx mice treated with the compound 22 compared to the mdx control mice, as emerges from FIGS. 11A and 11B. However, no difference in terms of fibrosis or cell infiltrate emerged, although the mdx mice treated with SEQ ID No.2 showed an increasing trend of the percentage of necrotic fibres compared to the other experimental conditions (FIG. 9C).

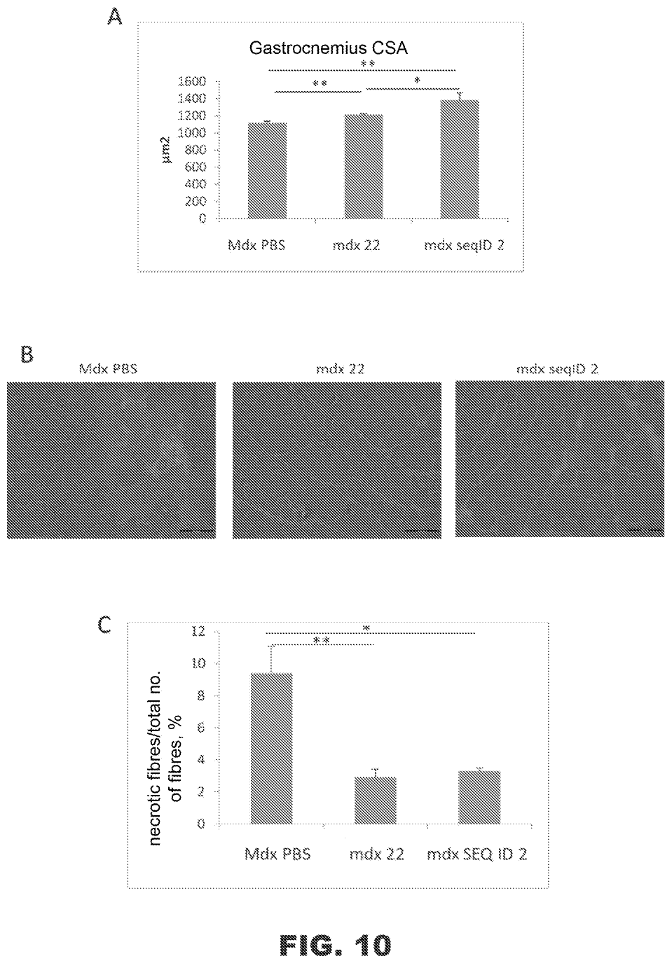

The histological analysis on sections of gastrocnemius muscle highlighted an increase in the mean cross-section area of the fibres in mdx mice treated with the compound 22, a reduction in the cell infiltrate and a drop in the number of degenerating myofibers compared to the control mice (FIG. 10A-C).

Surprisingly, the muscle that benefited most from the treatment with the compound 22 was the diaphragm, namely the most affected in the mdx mice. The treatment led to an increase in the mean cross-section area of the fibres, a reduction in the cell infiltrate and in the percentage of degenerating fibres (FIG. 11A-C).

### Immunofluorescence

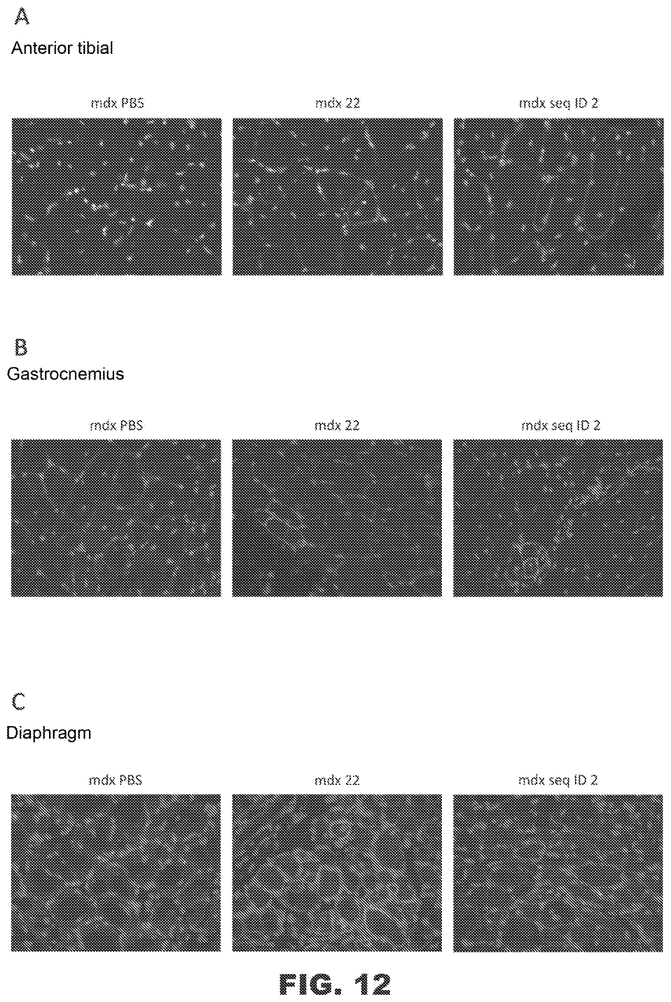

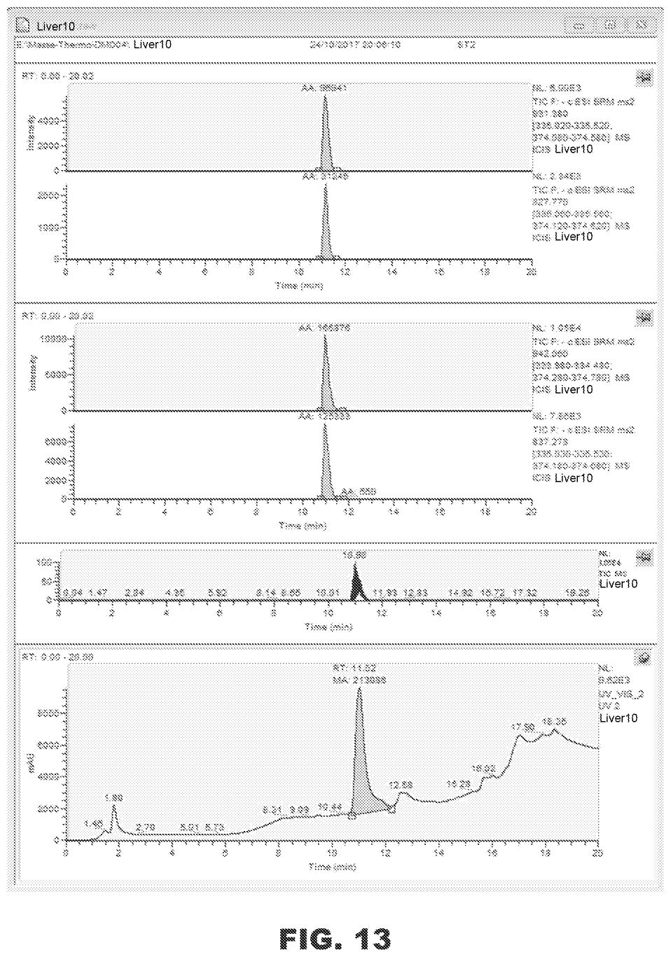

The immunofluorescence analysis for the dystrophin revealed the presence of some positive fibres scattered in sections of anterior tibial and gastrocnemius muscle of mdx mice treated with the conjugate 22 compared to the mdx control mice. However, by way of confirmation of the improvement at histological level, the dystrophin is produced and expressed to a greater extent in the diaphragm myofibers of mdx mice treated with the conjugate 22 (FIG. 12A-C).

### Quantification by Means of LC/MS/MS

The analyses designed for quantification of the oligonucleotides in mouse cell extracts (liver, kidney, gastrocnemius muscle, tibial muscle, diaphragm and heart) were carried out using a Thermo TSQ Quantum Access Max spectrometer interfaced with a Thermo Ultimate 3000 HPLC.

The cell tissues were used in the form of 5% tissue cell homogenates (50 mg per mL) digested with proteinase K and sterilized for 10 min under UV lamp before being frozen. The samples were kept at −20° C. until use.

In particular the compounds 22, 23, SEQ ID 1 and SEQ ID 2 were studied.

The pure oligonucleotides, used as standard, were kept in solid form, at −20° C. From these, work solutions were prepared at a concentration of 5.0 OD/mL, quantified at 260 nm and kept at 4° C. for up to six months (periodically controlled for UV and MS).

### Extraction Method

800 μL of sample were lyophilised and dissolved in 200 μL of water containing the Internal Standard (IS) and aqueous TEAB. The reconstituted solutions were chromatographed on two SPE cartridges (Oasis LHB 10 mg) and eluted with a known protocol (Nature medicine 2015, 21, 270-279). The extracted fractions were lyophilised and re-dissolved in 200 μL of solution A for HPLC-MS/MS analysis.

### HPLC Protocol

The tissue extracts and the aqueous samples were analysed with an X-Terra MS C18 2.5 μm, 4.6×50 mm column. Eluants used: Solution A 100 mM hexafluoropropanol, 8.6 mM triethylamine in water; Solution B MeOH. Flow 0.5 mL/min, Gradient: 0-3 min 100% A; 3-15 min linear variation up to 20% A, 80% B; 15-18 min 20% A, 80% B; 18-20 min linear variation up to 100% A, 22 or 24 min stroke end.

During the stroke the UV signals were recorded at 200 and at 600 nm and the PDA signal from 200 to 350 nm. Normally 30 μL of sample were injected.

### MS/MS Method

The conjugate 23 served as IS for analysis of the conjugate 22, whereas SEQ ID No.1 served as IS for quantification of the oligonucleotide SEQ ID No.2. For each compound the precursor→derivative fragmentations were identified that give rise to the most intense ions (normally those deriving from the precursors with eight charges (fragmentation B).

Fragmentations used: (transitions)

22-A 837.278→335.28+374.42

22-B 942.060→334.23+375.53

22-C sum of the two previous ones

SI-A 827.77→335.31+374.37

SI-B 931.38→335.27+374.33

SI-C sum of the two previous ones

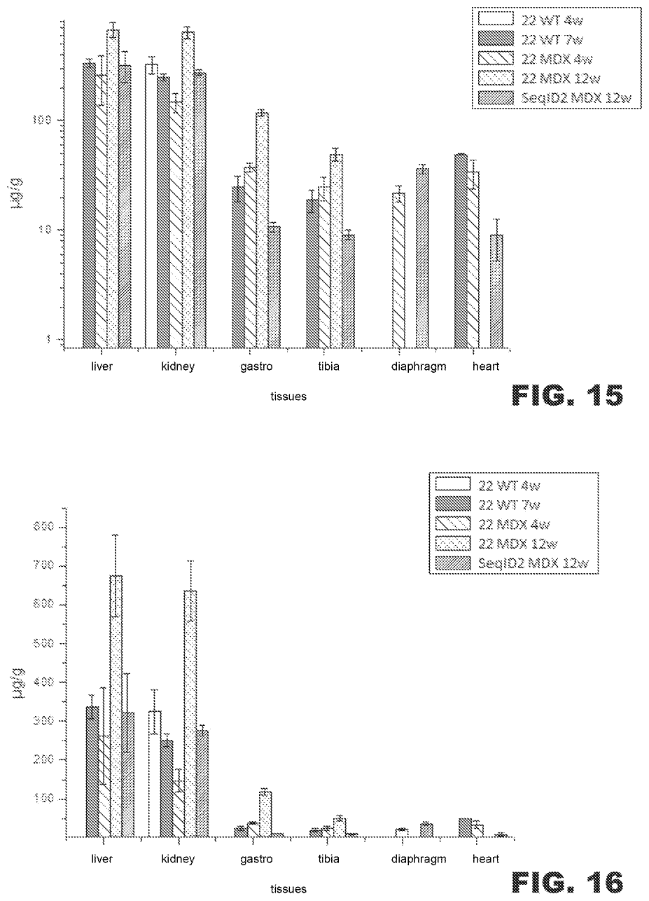

As an example, the following analysis is reported relative to a mix of conjugates 22 and 23 each at 0.05 OD/mL corresponding to 2.08 μg/ml (FIG. 15).

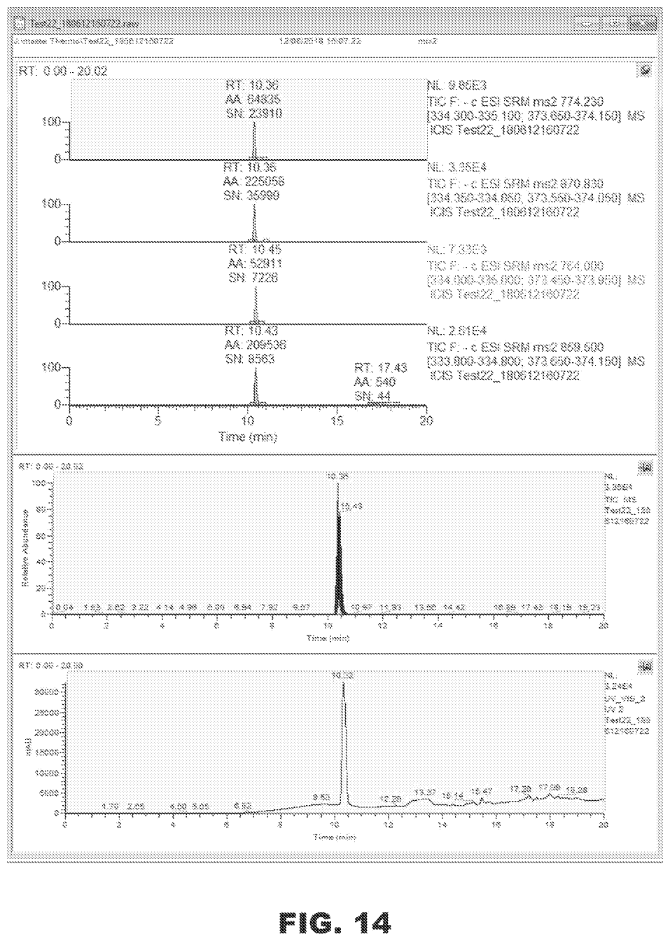

Similarly for the pairs SEQ ID 2 and SEQ ID 1 (SI): FIG. 14.

In this case the transitions used are the following:

SEQ ID 2-N A 859.5→334.3+373.9

SEQ ID 2-N B 764.0→334.5+375.7

SEQ ID 2-N C sum of the two previous ones

SI-A 870.83→334.6+373.9

SI-B 774.23→334.7+373.9

SI-C sum of the two previous ones

### Quantifications

The samples belonging to the different batches were analysed together in rapid succession, the analysis sequence contains numerous water samples to verify the absence of entrainment phenomena, and at least one complete calibration sequence. For each sequence, the analyses of some samples are repeated to verify analytical reproducibility. The quantifications are obtained by automatically integrating the areas of the relative signals with the help of the Thermo LC Quan software and visually inspected to check for possible interferences or accidental errors. The software calculates the ratio between the area of the preselected transition of the target oligonucleotide and that of the standard, transfers it to the calibration curve (normally a line) transforming the result into mg/g of tissue, on the basis of the parameters provided based on the quantity of internal standard added.

The presence of two different transitions and several ions collected allows numerous controls to be obtained on the analytical datum. In each analysis quality standards are added deriving from aqueous samples or cellular extracts treated only with the PBS (with known ratios between the oligonucleotides) to verify the correctness of the method used.

In the samples particularly rich in conjugate, successive analyses were performed in full-scan mode in search of possible recognisable ions, in particular those deriving from fragmentation of the conjugates used. In all the samples analysed important quantities of 5′UDC with one, two or three bases missing from the terminal 3′ were found. In the absence of standard and a consolidated procedure, said compounds were quantified based on the intensity of the ions observed, compared with those of the intact conjugate.

### Validations

The analyses were convalidated by analysing similar extracts of cell samples of mice treated only with PBS, in which known quantities of the oligonucleotides were added. The quantities measured by the analyses of said control samples (QC) were within the limit of 5% of the expected value.

### Cell Content

The quantity of intact sample found in the various tissues at 4, 7 and 12 weeks of treatment in wild type (WT) or genetically modified (MDX) mice is summarised in the following table 3 and in FIGS. 15 and 16.

TABLE 3

| | | | | | | | | | seqID | |
|---|---|---|---|---|---|---|---|---|---|---|
| | 22 in WT | | 22 in WT | | 22 in MDX | | 22 in MDX | | 2 in MDX | |
| | 4 weeks | | 7 weeks | | 4 weeks | | 12 weeks | | 12 weeks | |
| | μg/g | st dev | μg/g | st dev | μg/g | st dev | μg/g | st dev | μg/g | st dev |
| liver | — | — | 336.3 | 30.9 | 263 | 124.9 | 675 | 105 | 322 | 101 |
| kidney | 325 | 57.4 | 250.9 | 16 | 147 | 29.7 | 635 | 77 | 275 | 14 |
| Gastrocnemius | — | — | 24.7 | 6.5 | 37.6 | 3.7 | 118.4 | 9.4 | 10.75 | 1.03 |
| tibial | — | — | 18.8 | 4.3 | 24.6 | 6.1 | 49.5 | 6.7 | 9.08 | 0.93 |
| diaphragm | — | — | — | — | 21.8 | 3.6 | | | 36.14 | 3.53 |
| heart | — | — | 49.3 | 0.6 | 33.8 | 10 | | | 8.99 | 3.75 |

The exon skipping was evaluated by means of RT-PCR carried out with primers complementary to exons 20 and 26 of the murine dystrophin transcript able to amplify the fragment of 1098 pairs of bases corresponding to the complete transcript and 885 pairs of bases corresponding to the transcript without the exon 23 (FIG. 5A).

With the exception of the heart, in all the muscles analysed, the treatment with the compound 22 induced higher skipping levels than the oligonucleotide of SEQ ID No.2, with the highest skipping levels identified in the diaphragm (FIG. 5B).

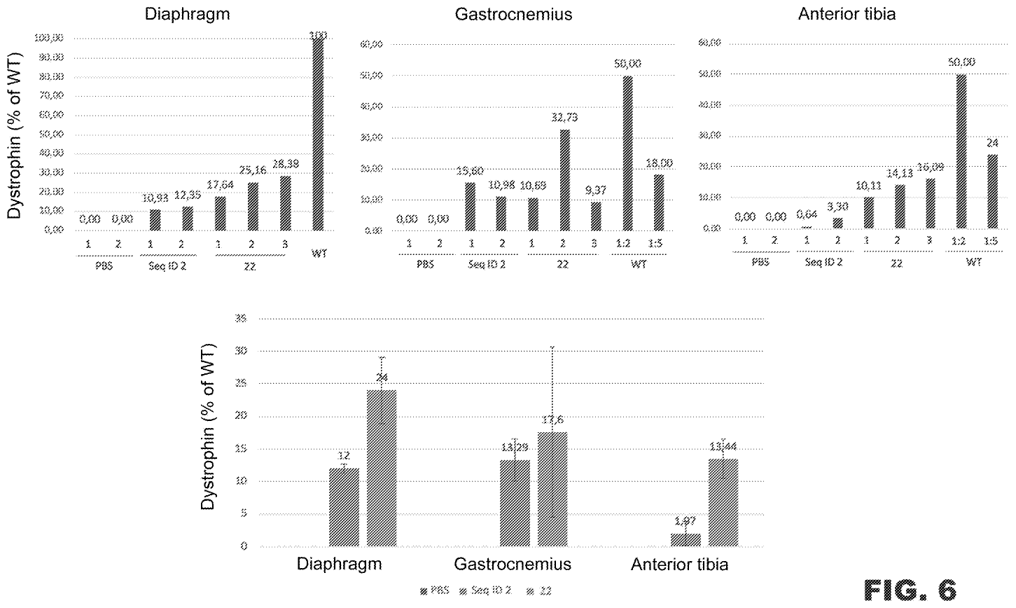

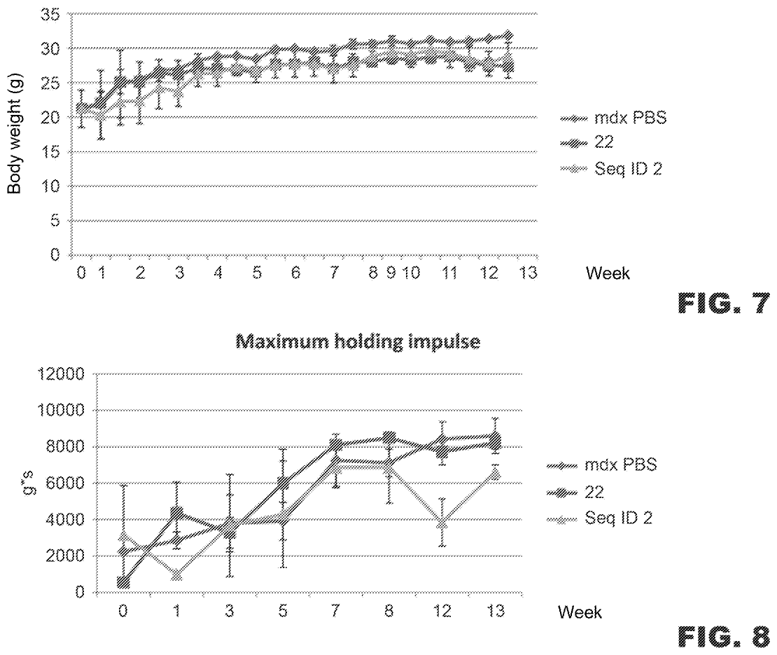

The muscles collected for semiquantitative analysis of the dystrophin by means of western blot were homogenized in RIPA buffer and protease inhibitors to be subsequently quantified. Thirty micrograms of proteins were mixed with NuPage LDS 4× buffer with the addition of 50 mM DTT, heated for two minutes to 85° C. before being loaded on a Novex 3%-8% Tris-Acetate gel and migrated for 70 minutes at 150V. The proteins were then transferred onto PVDF membranes by means of iBLOT system at 70V for 7 minutes and hybridized with antibodies against the carboxyterminal region of the dystrophin (NCL-DYS2) and against the alpha-actinin as loading control. The quantification of the protein by means of western blot highlighted an increase in dystrophin produced in all the treatments and a higher quantity in the mice treated with the compound 22 (FIG. 6).

With the exception of the heart, all the treated muscles analysed showed renewed dystrophin expression, completely absent in the control mice (injected with PBS). The treatment with 22 restored a greater expression of dystrophin than the non-conjugated oligonucleotide SEQ ID No.2.

1. A conjugate of oligonucleotides and bile acid derivatives having the structure (I), (II) or (III) 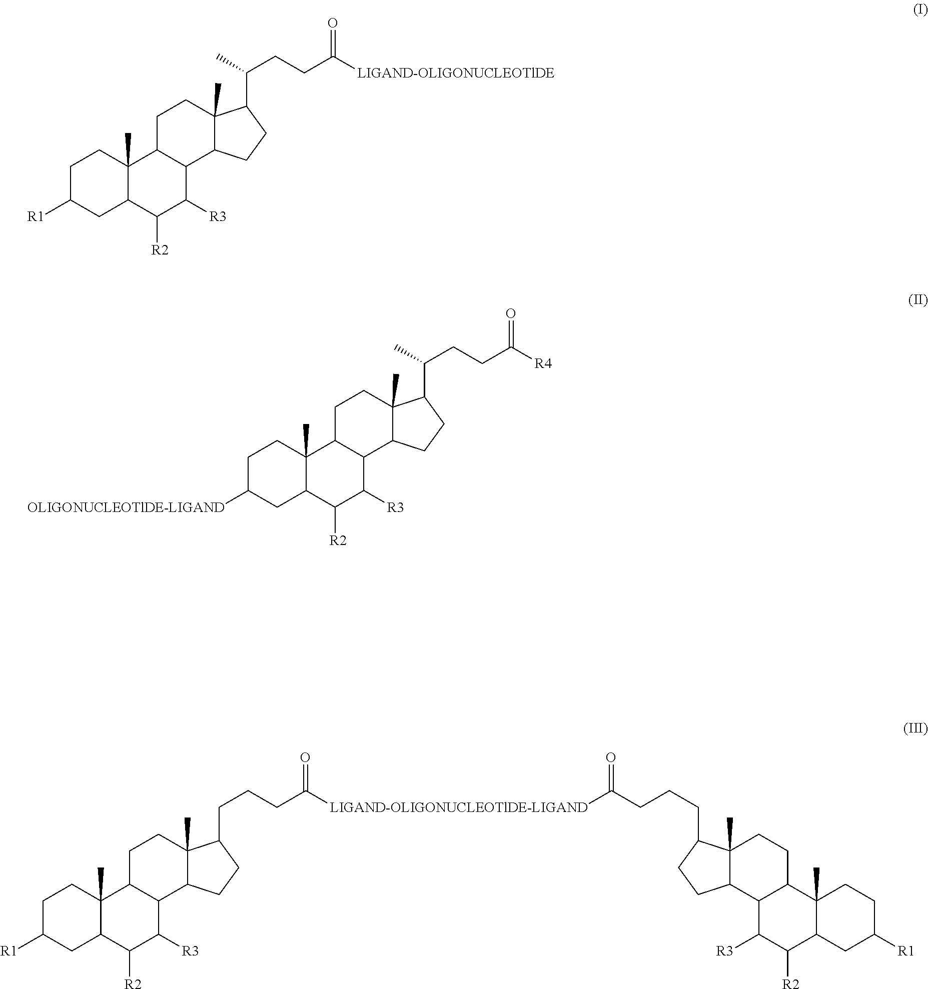
wherein
R₁, R₂ and R₃ are independently selected from the group consisting of H, OH, NH₂, —NHC(O)R₅, and C(O)R₅;
R₄ is selected from the group consisting of OH, NH₂, —NH(C₁₋₆alkyl)SO₃H;
R₅ is selected from the group consisting of a saturated or partially unsaturated, linear or branched C₃-C₃₁ aliphatic hydrocarbon;
the ligand has formula (IV) or (V)
a) —X—Y—NH(C₂₋₁₀alkyl)OP(═O)(Z)O— (IV)
wherein
X binds the bile acid residue and is selected from the group consisting of bond, —NHC(O)(C₂₋₁₀alkyl)C(O) and —NH(C₂₋₁₀alkyl(NHR₆))C(O)— where R₆ is selected from the group consisting of —H and 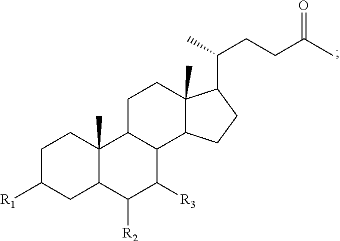
Y is selected from the group consisting of bond and NH(C₂₋₁₀alkyl)OC(O);
Z is selected from the group consisting of S⁻ and O⁻ and
the group OP(═O)(Z)O— binds the oligonucleotide or
b) 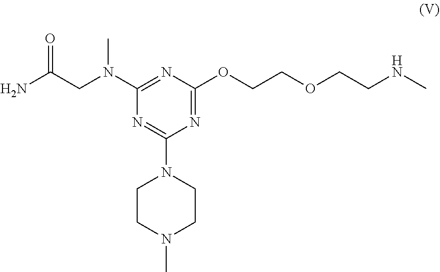 [SMILES: CNCCOCCOc1nc(N(C)CC(N)=O)nc(N2CCN(C)CC2)n1]
where the piperazine residue binds the oligonucleotide and the amine residue binds the bile acid residue.

2. The conjugate according to claim 1, characterised in that R₁ is selected from the group consisting of OH, NH₂, and —NHC(O)R₅.

3. The conjugate according to claim 1, characterised in that R₁ is selected from the group consisting of OH, NH₂, —NHC(O)(CH₂)₃(CH═CH—CH₂)₅CH₃, and —NHC(O)(CH₂)₂(CH═CH—CH₂)₆CH₃.

4. The conjugate according to claim 1, characterised in that R₃ is selected from the group consisting of —H and —OH.

5. The conjugate according to claim 1, characterised in that R₄ is selected from the group consisting of OH and —NH(C₂H₄)SO₃H.

6. The conjugate according to claim 1, characterised in that the ligand is selected from the group consisting of

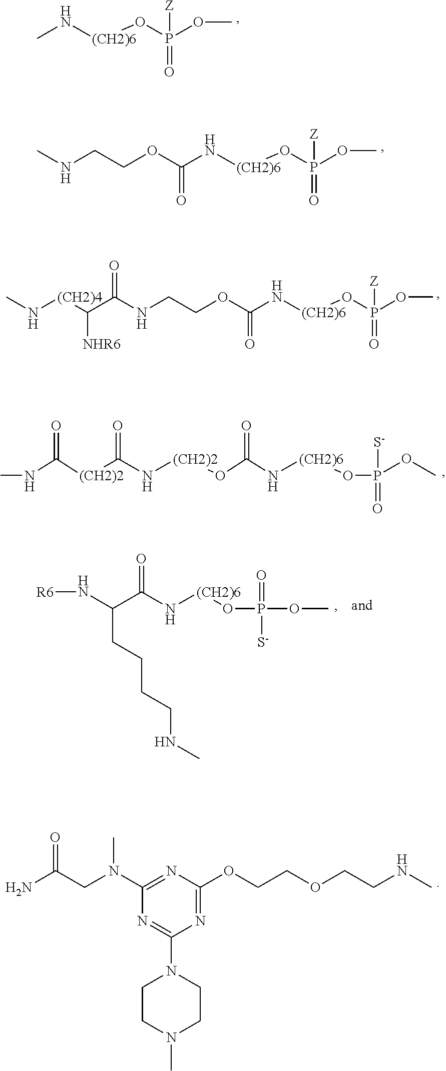

7. The conjugate according to claim 1, characterised in that said oligonucleotide is an antisense oligonucleotide specific for a splicing sequence in an mRNA of interest.

8. The conjugate according to claim 7, characterised in that said oligonucleotide is selected from the group consisting of SEQID No.1, SEQID No.2, SEQID No.3, SEQID No.4, SEQID No.5 and SEQID No.6.

9. The conjugate according to claim 1, selected from the group consisting of:

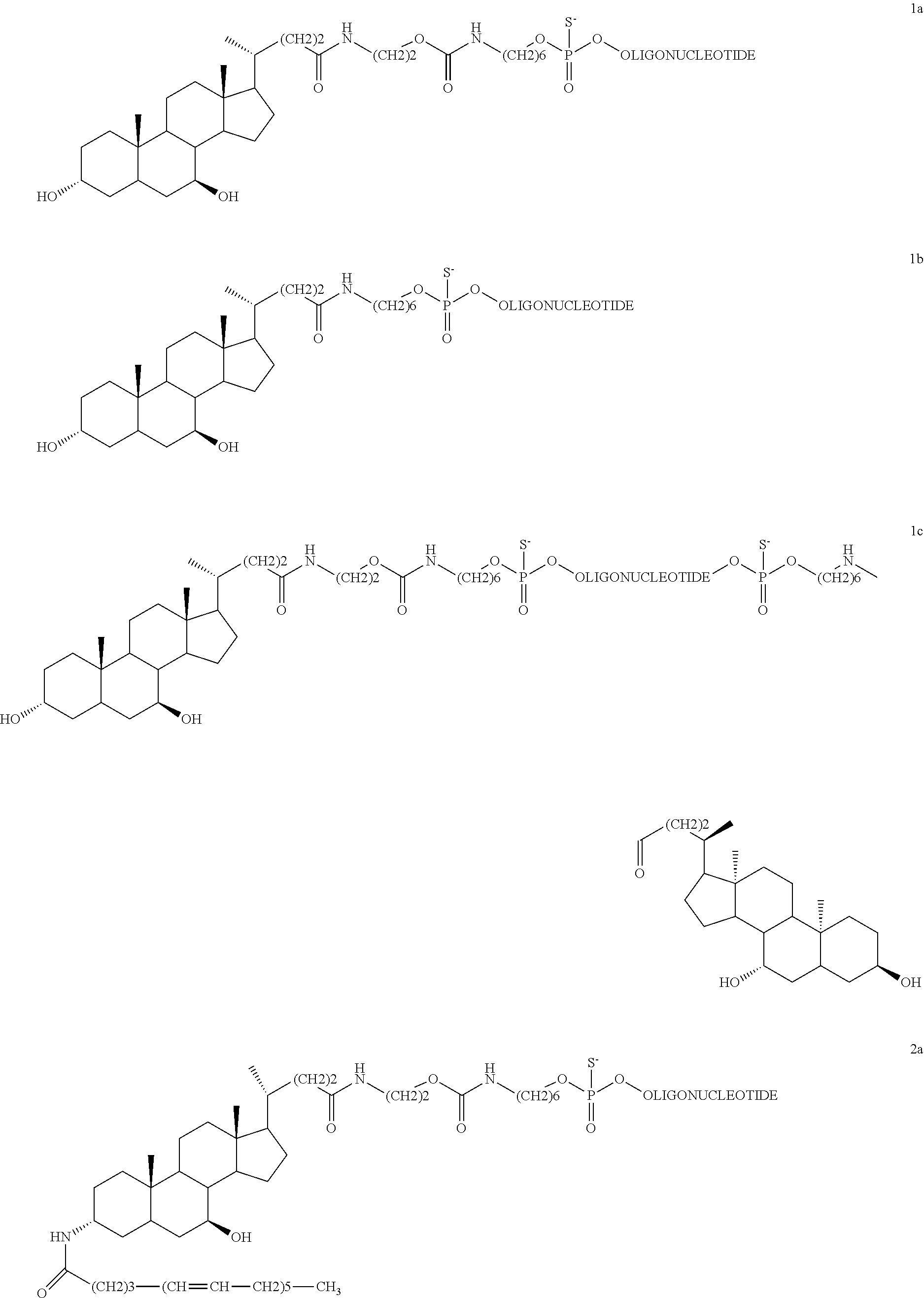

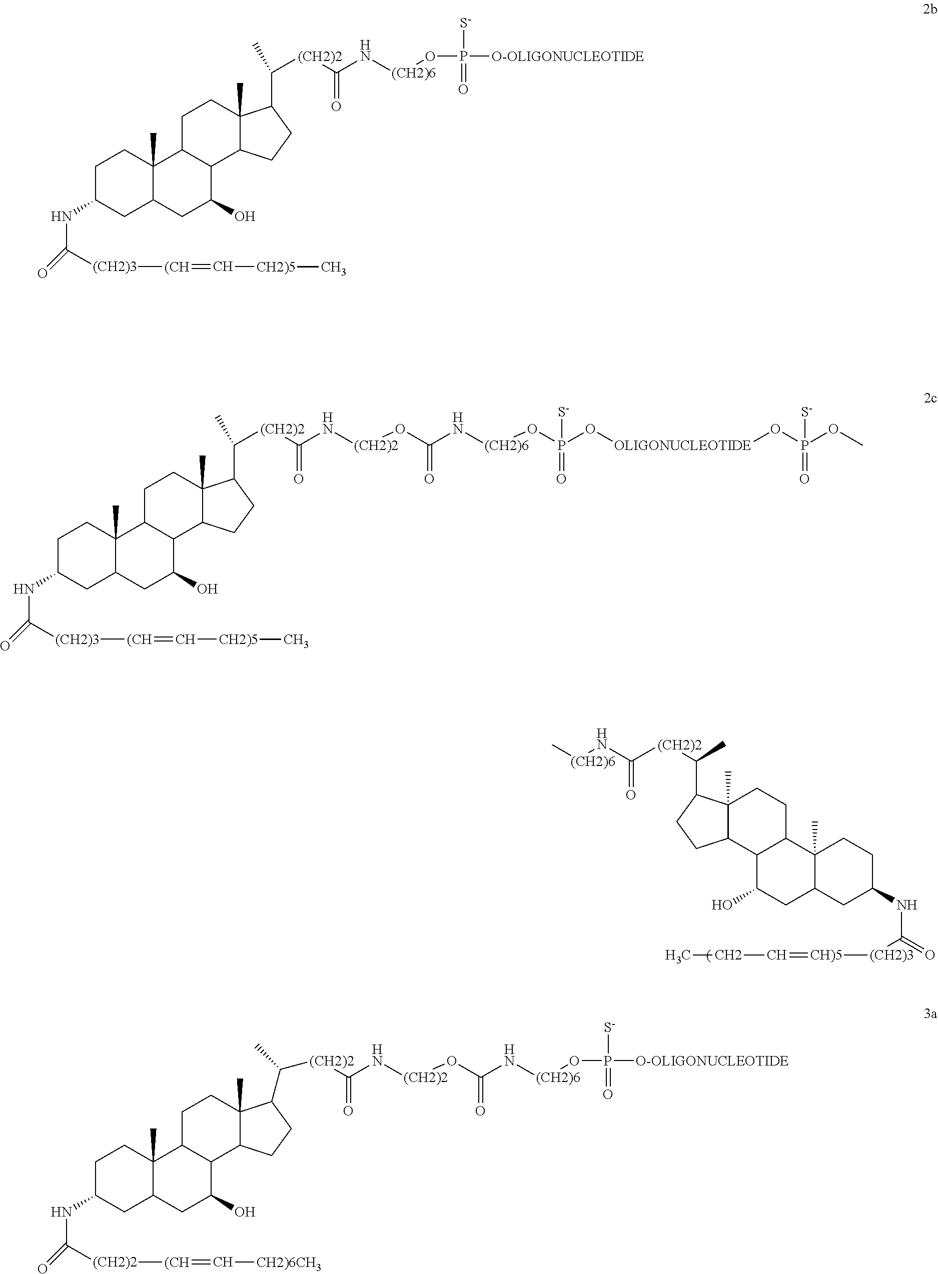

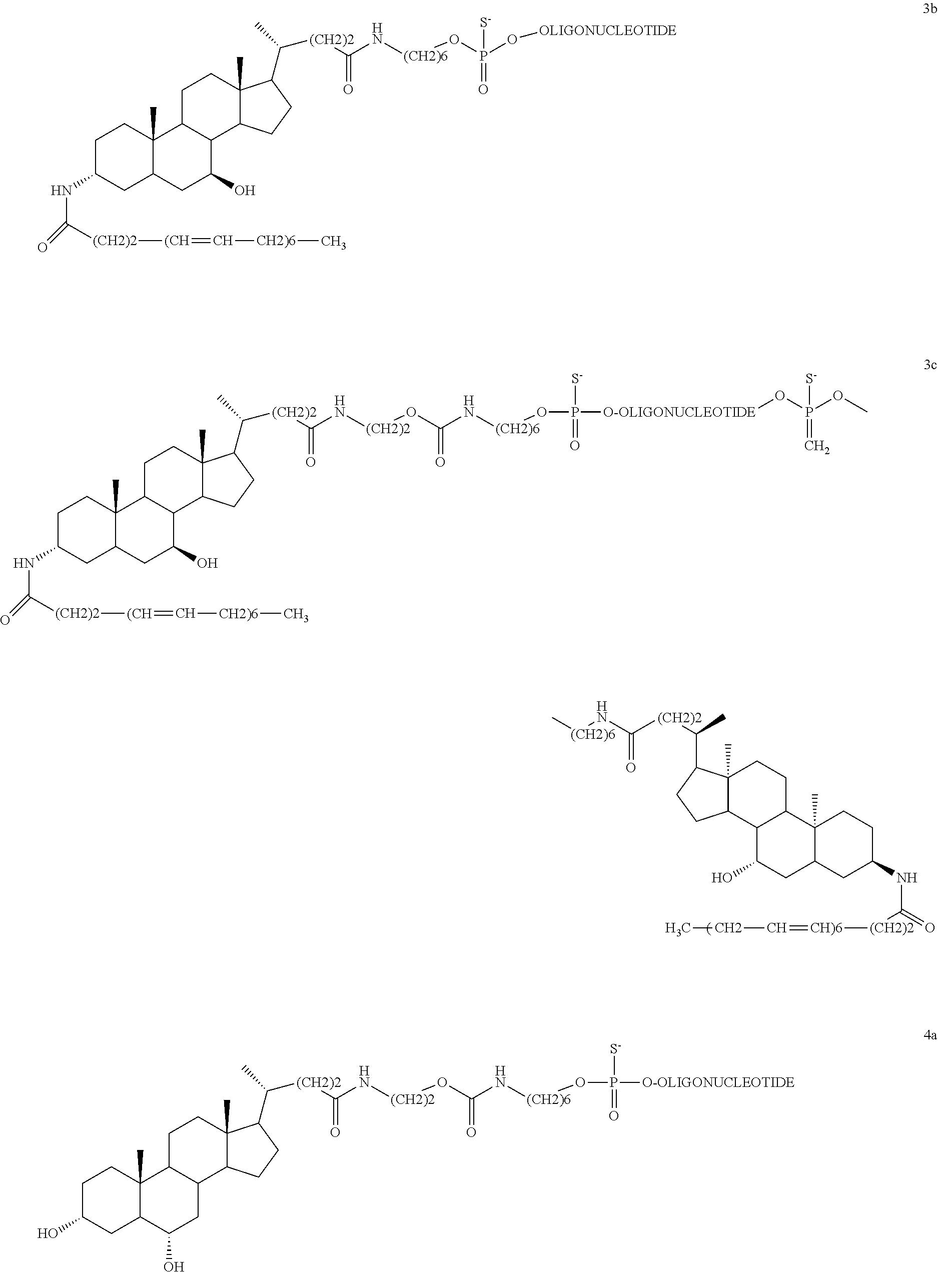

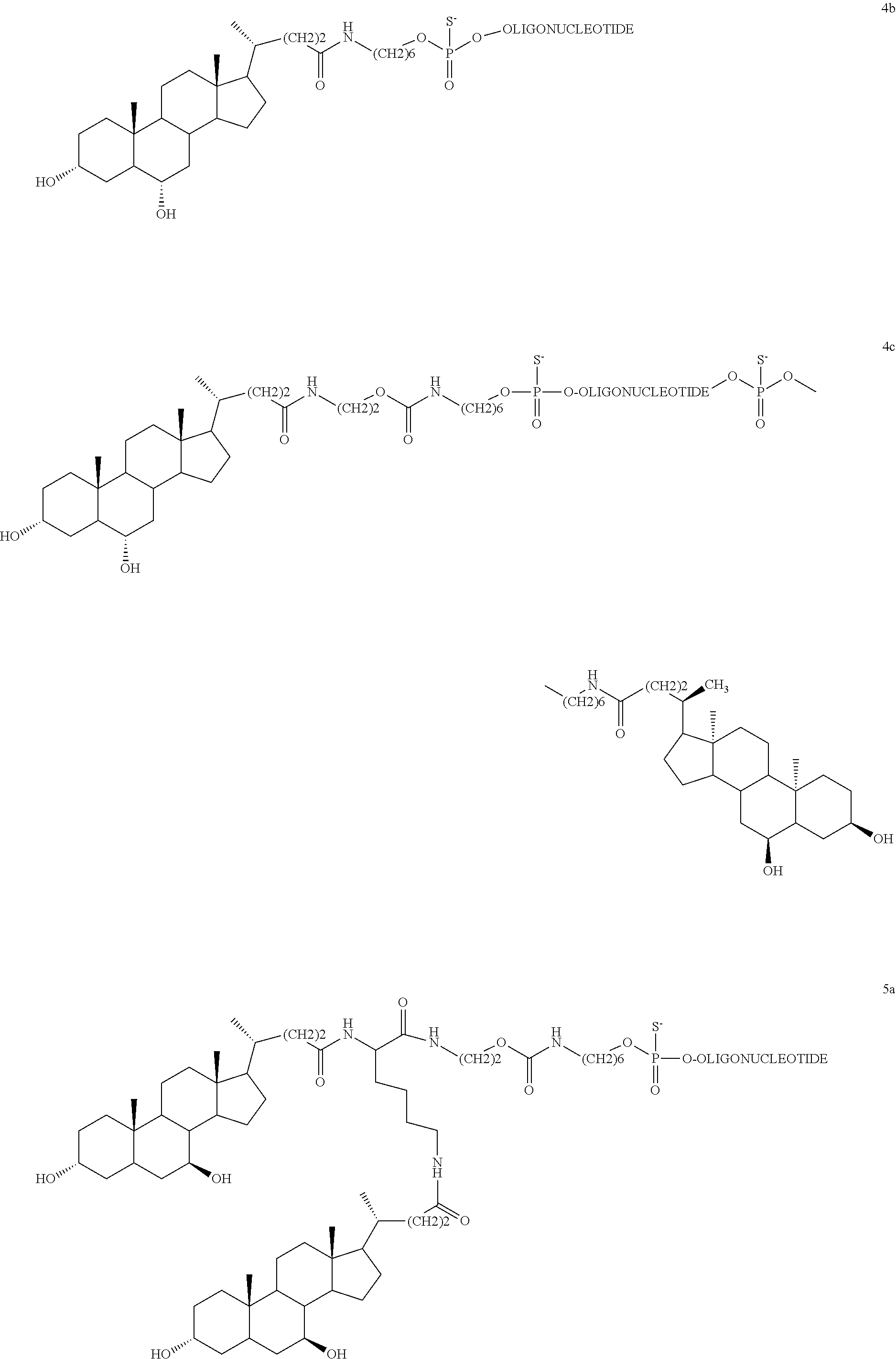

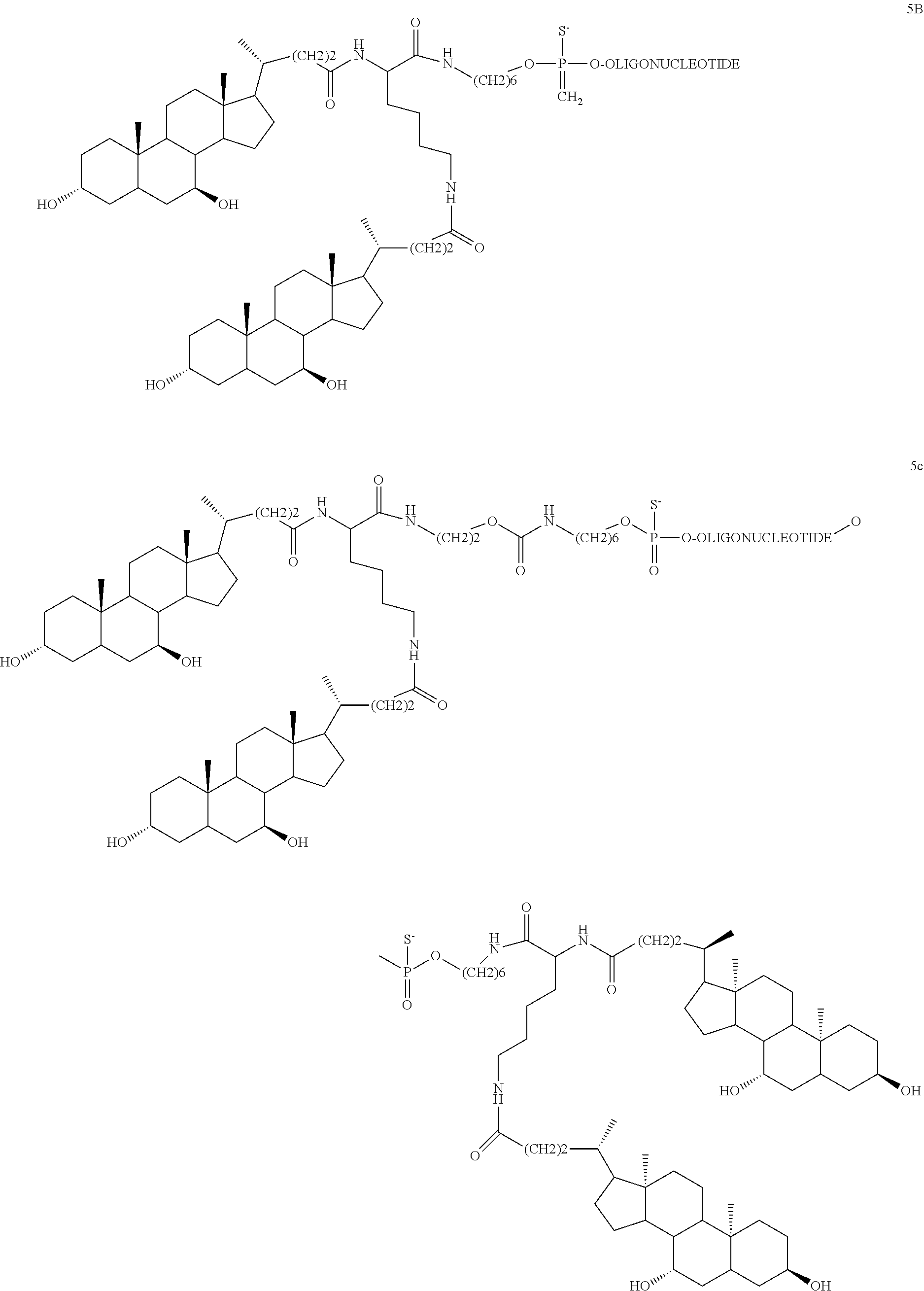

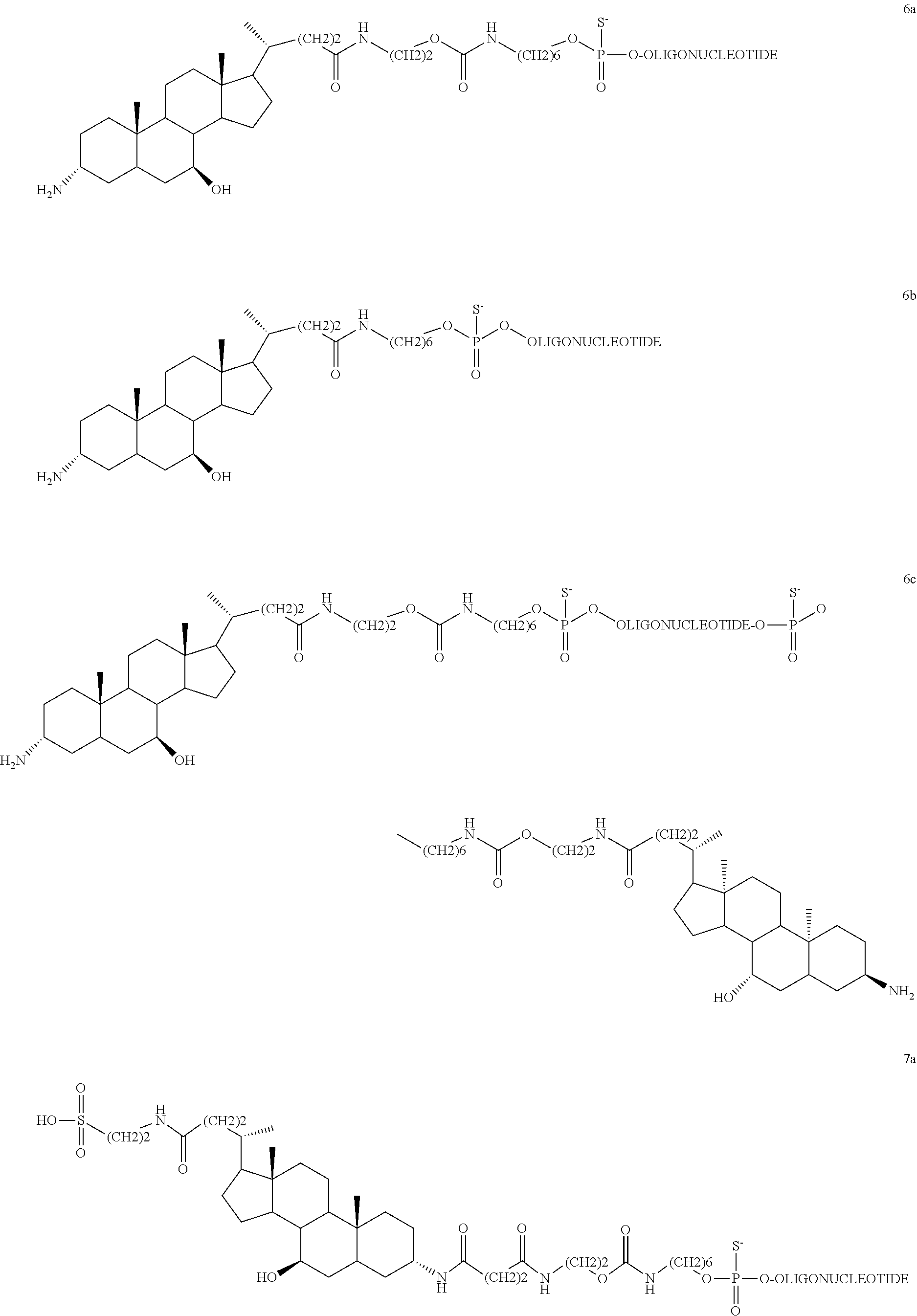

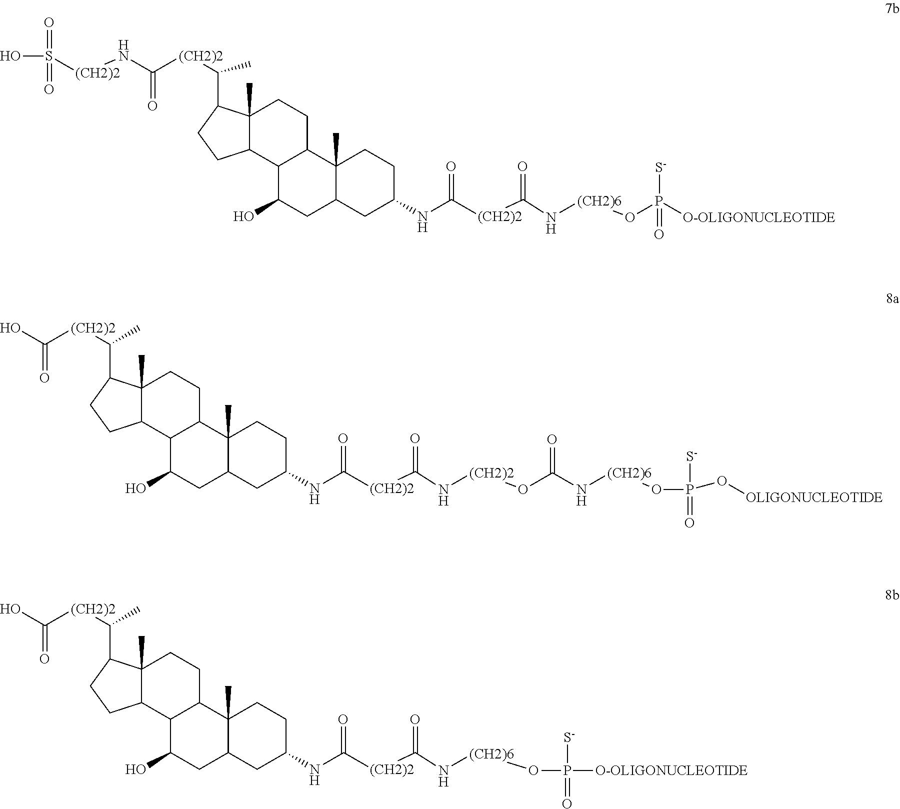

10. The conjugate according to claim 8, selected from the group consisting of:

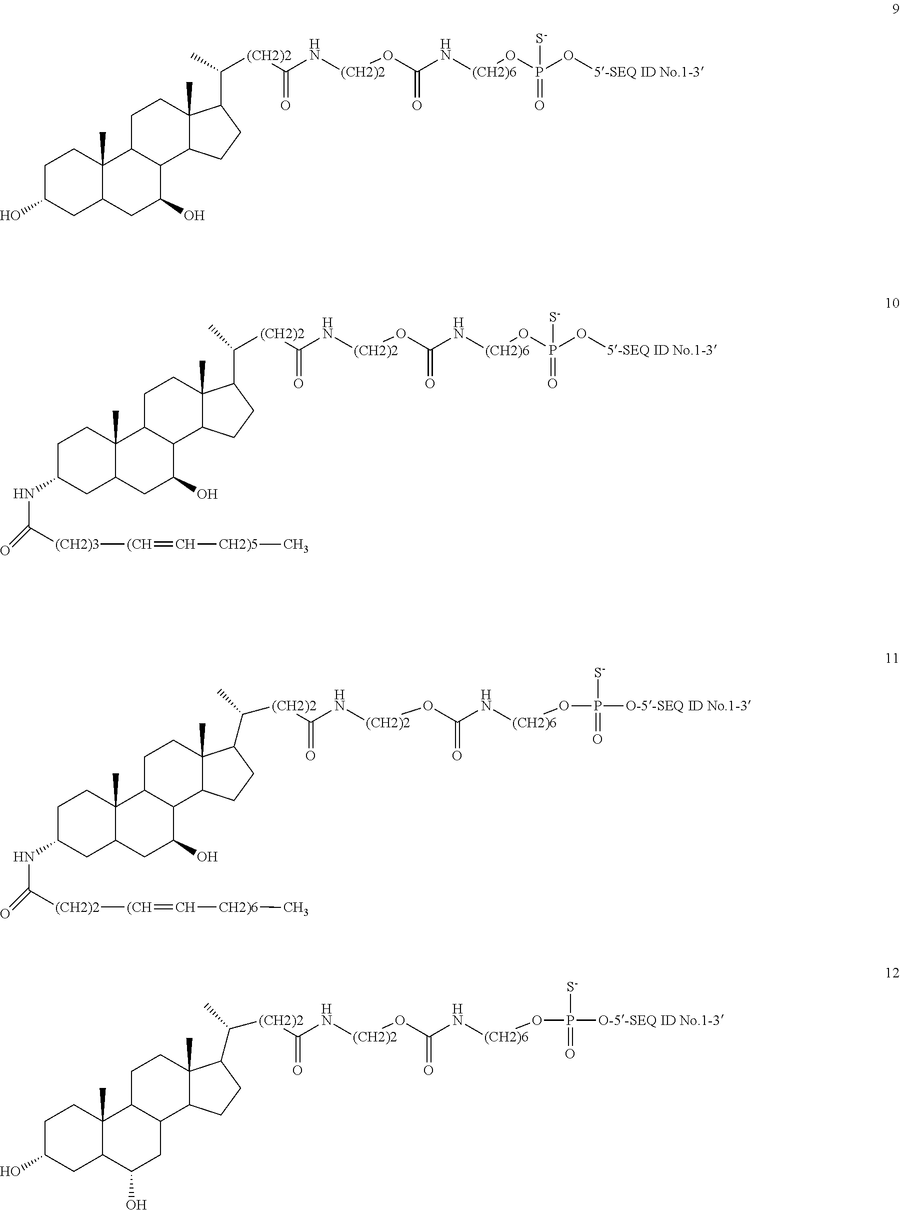

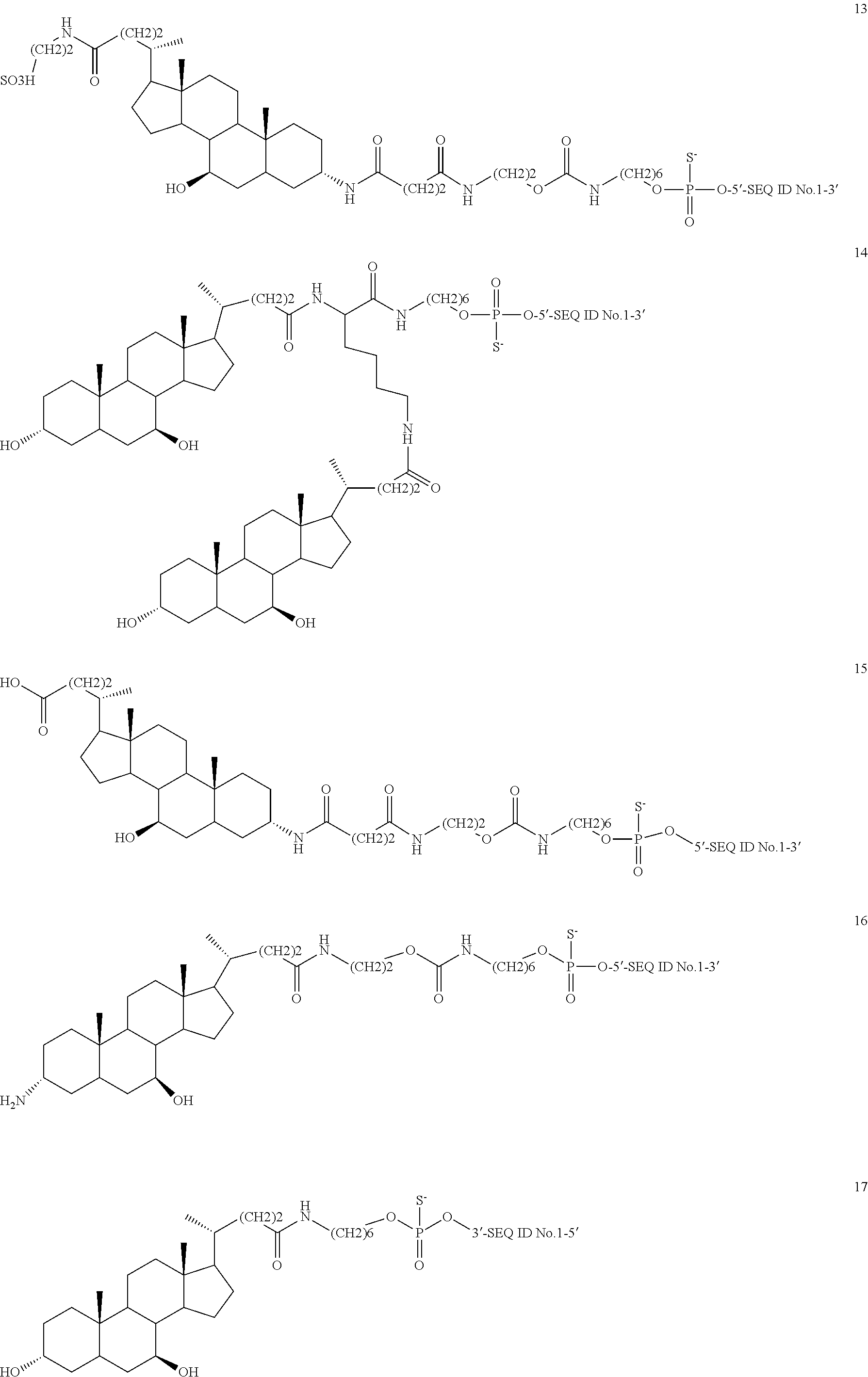

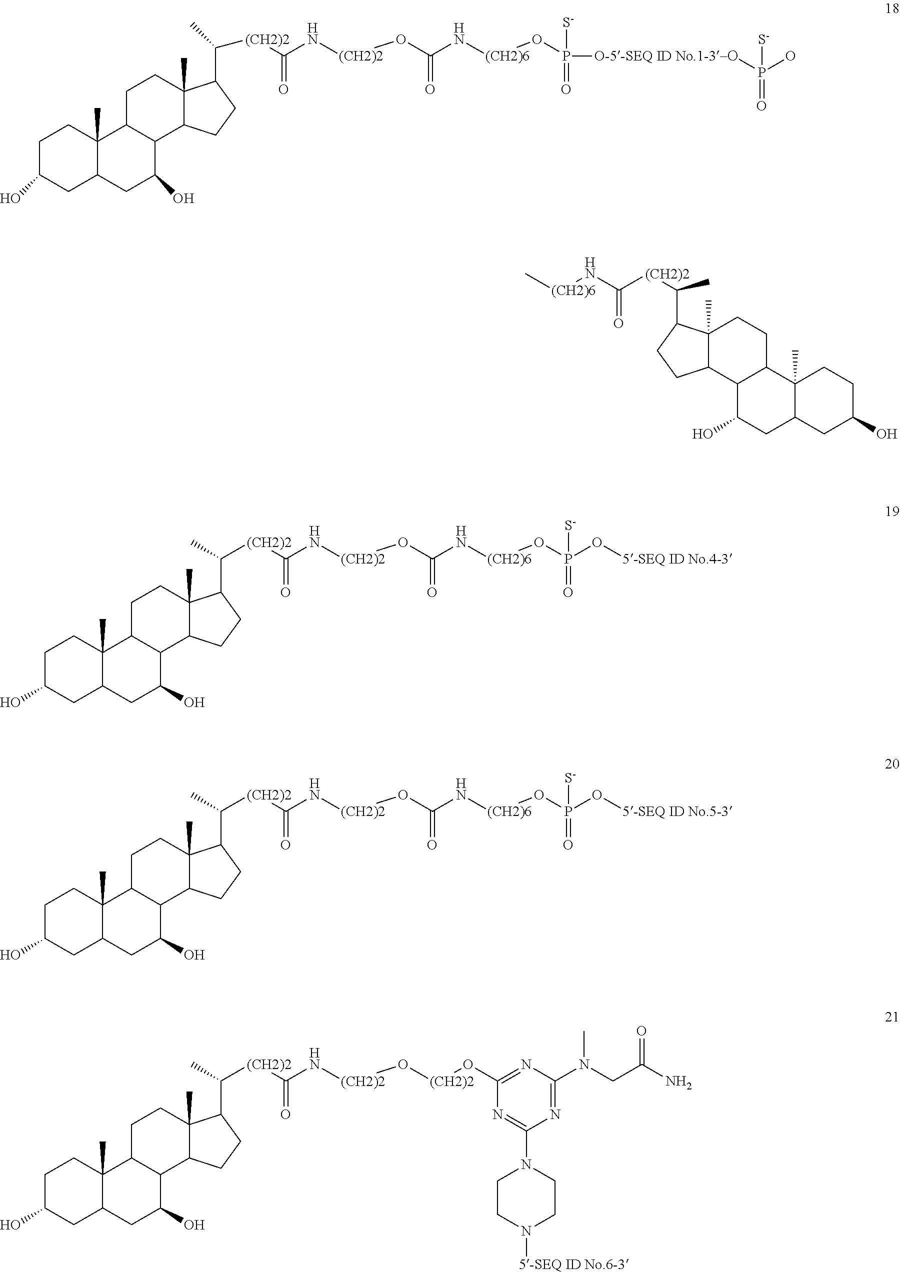

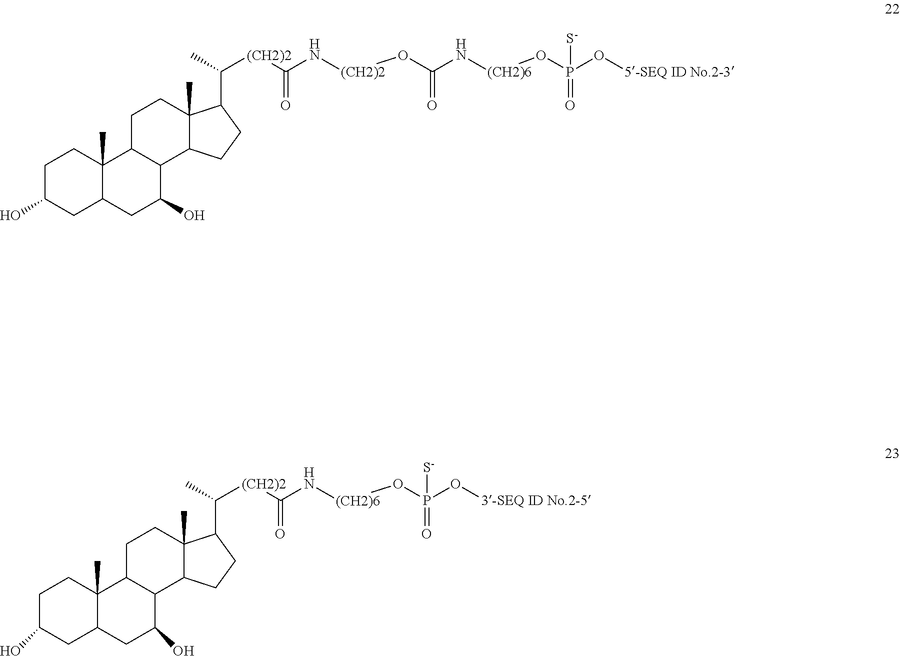

11. A pharmaceutical composition comprising the conjugate according to claim 1.

12. A method comprising administering to a subject in need thereof the conjugate according to claim 1 as a medicament.

13. A method for improving exon skipping in an mRNA of interest comprising administering to a subject in need thereof the conjugate according to claim 7.

14. A method of treating a disease comprising administer to a patient in thereof the conjugate according to claim 7, wherein the disease is selected from the group consisting of Duchenne dystrophy, Bardet-Biedel syndrome, beta thalassemia, cancer, cystic fibrosis, factor VII deficiency, familial dysautonomia, Fanconi anaemia, haemophilia A, propionic acidemia, retinitis pigmentosa, ataxia telangiectasia, congenital disorders of glycosylation, congenital adrenal insufficiency, Fukuyama congenital dystrophy, growth hormone insensitivity, BH4 deficiency hyperphenylalaninemia, Hutchinson-Gilford progeria, megalencephalic leukoencephalopathy with subcortical cysts, methylmalonic aciduria, myopathy with lactic acidosis, myotonic dystrophy, neurofibromatosis, Niemann-Pick disease type C, Usher syndrome, afibrinogenemia, ocular albinism type 1, Alzheimer's disease, tauopathies, spinal muscular atrophy, atherosclerosis, inflammatory diseases, muscular atrophy diseases, spinocerebellar ataxia type 1, dystrophic epidermolysis bullosa, and Miyoshi myopathy.

15. The method according to claim 14, characterised in that said disease is Duchenne dystrophy.


SEQUENCE LISTING (6 sequences)

SEQ ID NO: 1 - RNA, 20 nt, Artificial Sequence
  PRO051
  misc_feature (1)..(20): /note=“on each ribose nucleotide modified at 2′ with a methoxy”
ucaaggaagauggcauuucu

SEQ ID NO: 2 - RNA, 20 nt, Artificial Sequence
  M23D short
  misc_feature (1)..(20): /note=“on each ribose nucleotide modified at 2′ with a methoxy”
ggccaaaccucggcuuaccu

SEQ ID NO: 3 - RNA, 25 nt, Artificial Sequence
  MD23D
  misc_feature (1)..(25): /note=“on each ribose nucleotide modified at 2′ with a methoxy”
ggccaaaccucggcuuaccugaaau

SEQ ID NO: 4 - RNA, 26 nt, Artificial Sequence
  Long
  misc_feature (1)..(26): /note=“on each ribose nucleotide modified at 2′ with a methoxy”
guuuucuuuugaacaucuucucuuuc

SEQ ID NO: 5 - RNA, 30 nt, Artificial Sequence
  H2A
  misc_feature (1)..(30): /note=“on each ribose nucleotide modified at 2′ with a methoxy”
ccauuuugugaauguuuucuuuugaacauc

SEQ ID NO: 6 - RNA, 20 nt, Artificial Sequence
  PMO
  misc_feature (1)..(20): /note=“on each ribose nucleotide modified at 2′ with a morpholino”
ucaaggaagauggcauuucu